(19) United States
(12) Patent Application Publication
(10) Pub. No.: US 2006/0030571 A1
(43) Pub. Date: Feb. 9, 2006

(54) Ecteinascidins

(76) Inventors: Kenneth L. Rinehart, Urbana, IL (US); Ryuichi Sakai, Yokohama (JP)

Correspondence Address: MORGAN & FINNEGAN, L.L.P., 3 WORLD FINANCIAL CENTER, NEW YORK, NY, 10281-2101, US

(21) Appl. No.: 11/132,466

(22) Filed: May 18, 2005

Related U.S. Application Data
(63) Continuation of application No. 10/406,997, filed on Apr. 2, 2003, now abandoned.
Application No. 10/406,997 is a continuation of application No. 09/949,051, filed on Sep. 7, 2001, now abandoned.
Application No. 09/949,051 is a continuation of application No. 09/546,877, filed on Apr. 10, 2000, now abandoned.
Application No. 09/546,877 is a continuation of application No. 08/198,449, filed on Feb. 18, 1994, now abandoned.

(51) Int. Cl. A61K 31/519; C07D 491/14

(52) U.S. Cl. 514/257; 544/240

(57) ABSTRACT

The present invention is directed to several newly discovered ecteinascidin (Et) species, designated herein as Et 731, Et 745B, Et 815, Et 808, Et 596, Et 597, Et 583, Et 594 and a synthetic derivative of Et 594, N-Acetyl Et 597. The physical properties of these compounds, their preparation and bioactivities are also reported.

### BACKGROUND OF THE INVENTION

The ecteinascidins (herein abbreviated Et or Et's) are exceedingly potent antitumor agents isolated from the marine tuinicate Ecteinascidia turbinata. In particular, Et's 729, 743 and 722 have demonstrated promising efficacy in vivo, including activity against P388 murine leukemia, B16 melanoma, Lewis lung carcinoma, and several human tumor xenograft models in mice (see Reference 1d, cited below). The antitumor activities of Et 729 and Et 743 have been evaluated by the NCI and recent experiments have shown that Et 729 gave 8 of 10 survivors 60 days following infection with B16 melanoma. In view of these impressive results, the search for additional ecteinascidin compounds continues.

### SUMMARY OF THE INVENTION

The present invention is directed to the discovery of several additional ecteinascidin species, the structures of which provide evidence for the C units, the most unusual structural units present in the ecteinascidin family of compounds. An assignment of the absolute configuration of the. Et's C-unit as well as structures and bioactivities of other new Et analogues are also presented herein.

The structures of the new Et's are as shown in Chart I below:

CHART I

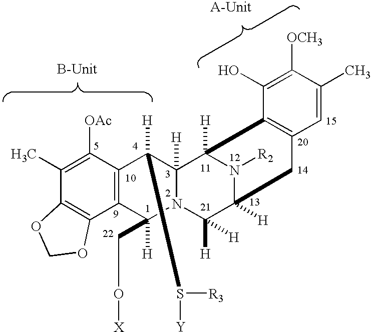

C-Units

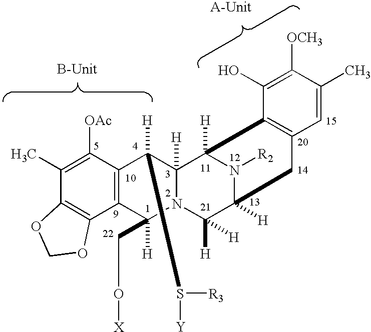

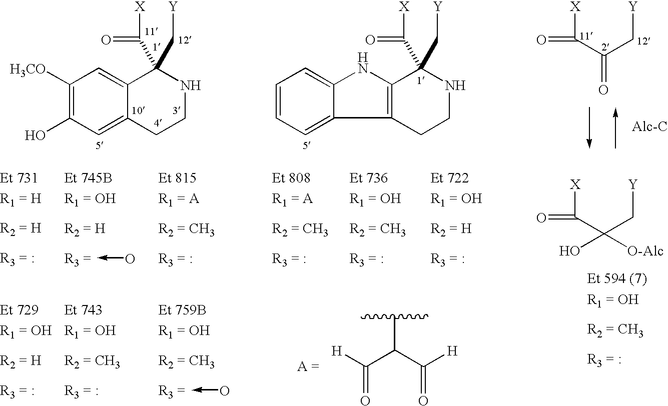

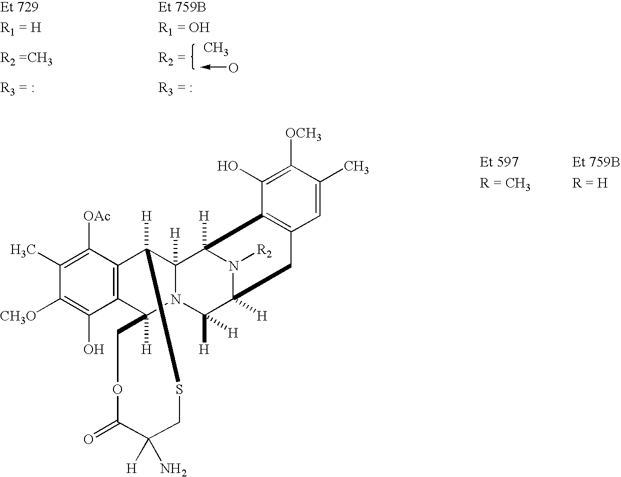

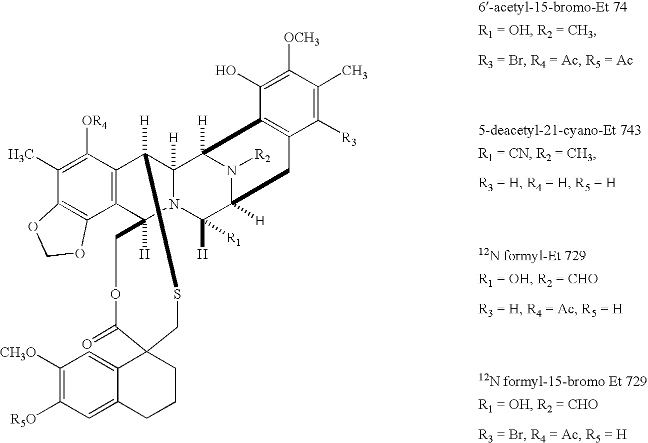

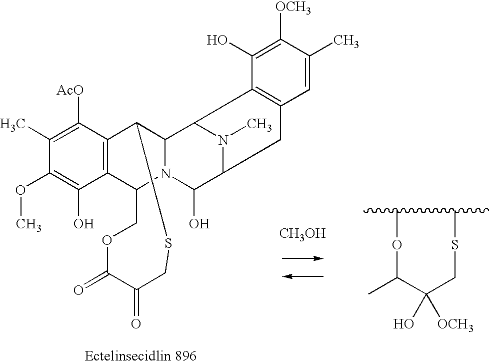

The new ecteinascidin compounds shown above have been found to possess the same activity profile as the known ecteinascidin compounds, and as such they will be useful as therapeutic compounds, e.g., for the treatment of mammalian tumors including melanoma, lung carcinoma, and the like. The dosages and routes of administration will vary according to the needs of the patient and the specific activity of the active ingredient. The determination of these parameters is within the ordinary skill of the practicing physician.

### BRIEF DESCRIPTION OF THE DRAWINGS

FIGS. 1A and 1B respectively show the ¹H NMR spectra for Et 731 and Et 745.

FIGS. 2A(1) and 2A(2) respectively show the ¹H NMR spectra for Et 745B and Et 759B.

FIG. 2B is the ¹³C NMR spectrum for Et 745B.

FIG. 3 illustrates the FABMS/CID/MS data-for Et 745B.

FIG. 4 is the ¹H NMR spectrum of Et 815, recorded in CD₃OD.

FIG. 5 illustrates the FABMS/CID/MS spectrum for the molecular: ion of Et 815.

FIGS. 6A and 6B respectively show the ¹H NMR spectra of Et 808 and Et 736.

FIG. 7 illustrates the FABMS/CID/MS data for Et 808.

FIG. 8 is the ¹H NMR spectrum of Et 597.

FIG. 9 illustrates the ¹H COSY spectrum of Et 597.

FIG. 10 illustrates the FABMS/CID/MS data for Et 597.

FIGS. 11A and 11B respectively show the ROESY NMR spectra for Et 597-monoacetate.

FIG. 12 shows the GC trace obtained by injection of a derivatized sample of Et 597, and of a D,L-mixture of TFA-Cys-OMe, showing that the Cys in the derivatized sample coelutes with the L-isomer of the standard mixture.

FIG. 13 is the ¹H NMR spectrum of Et 583.

FIGS. 14A and B, respectively show the FABMS spectra of Et 594 in glycerol, without oxalic acid and with oxalic acid.

FIG. 15 is the FABMS/CID/MS spectra of the methanol adduct of Et 594.

FIG. 16 is the ¹H NMR spectrum of Et 594, recorded in CD₃OD.

FIG. 17, trace lines A and B, respectively show the CD data for Et 597 and Et 743.

FIGS. 18-20 respectively show FABMS, FABMS/CID/MS and FABMS data for Et 596 and derivative compounds thereof.

### DETAILED DESCRIPTION OF THE PREFERRED EMBODIMENTS

Specimens of Ecteinascidia turbinata collected from the coast of Puerto Rico in August 1989 (PR-I), July 1990 (PR-II), August 1991 (PR-III) and September 1992 (ET-I) were extracted in the laboratory of Professor K. L. Rinehart at the University of Illinois, Urbana-Champaign, Ill. The isolation of bioactive components from PR-I and PR-II has previously been described (see References 1 and 2, cited below).

Newer specimens, PR-III and ET-I, were recently extracted to afford the previously known ecteinascidins species; Et's 729, 743, 722, 736 and other analogues, including Et 743-N¹²-oxide (Et759A), whose crystal structure was recently published (see Reference 2, cited below). Along with these previously described Et's, seven new ecteinascidins were isolated from the PR-III and ET-I extracts.

The present invention is thus directed to the isolation, structure determination, and cytotoxicities of these new Et species and Et-analogues.

A sample of E. turbinata (PR-III, 102 Kg) was collected in August of 1991 off the coast of Puerto Rico, at latitude 17°59′, longitude 67°5′, and at a depth of approximately 1-2 meters. Extraction and separation of the bioactive components were carried out using a bioassay guided scheme, to afford Et's 743 (123 mg), 729 (58.5 mg) and the new Et's 731 (4.85 mg), 745B (5.99 mg), 815 (358 mg), and 808 (0.8 mg).

A fresh sample of the tunicate (ET-I, 300 Kg) collected in September of 1992 from off the coast of Puerto Rico, was stored frozen and was similarly processed to afford Et 729 (2.0 mg) and the new Et 597 (1.7 mg).

Extraction of another batch of tunicate (about 100 Kg) collected in 1992-1993 from off the coast of Puerto Rico, gave the new Et 583 (1.432 mg) and Et 594 (1.20 mg) and an additional amount of Et 597 (1.45 mg).

Structure of Et 731

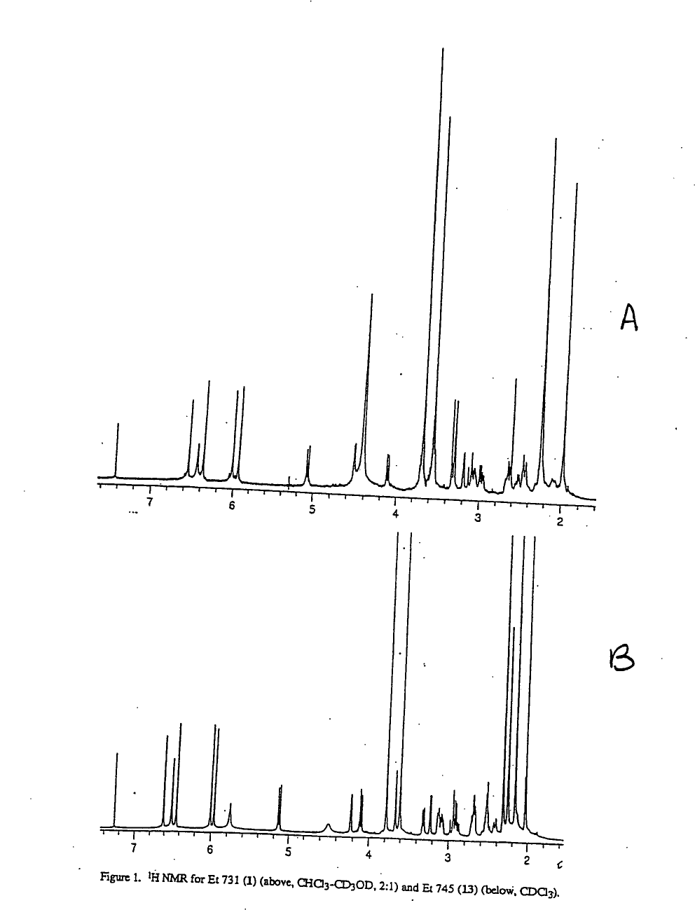

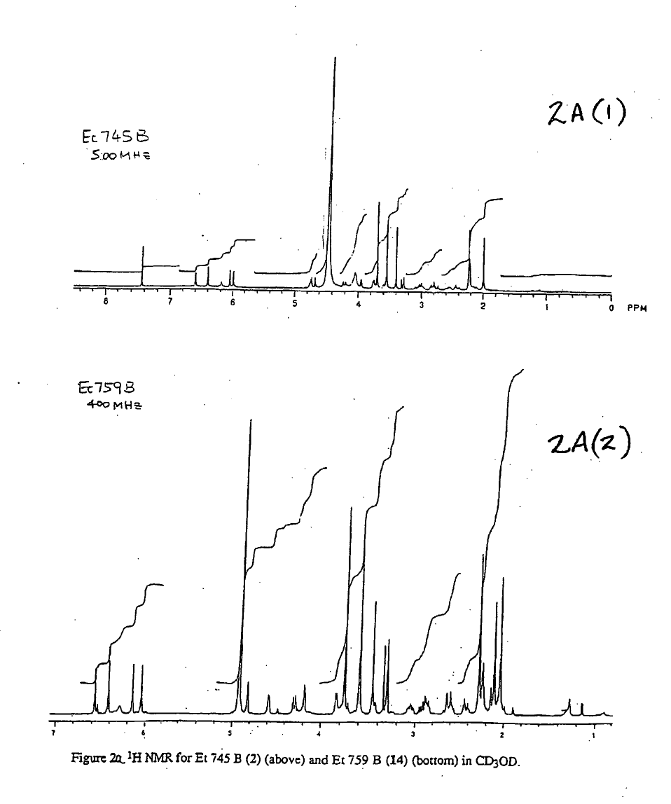

The molecular formula of Et 731, C₃₈H₄₁N₃O₁₀S, was assigned based on high resolution positive ion FABMS data for m/z 732 (M+H)⁺ and a negative FABMS ion at m/z 730 (M−H)⁻. A ¹H NMR spectrum of Et 731 had spectral characteristics illustrated in FIG. 1, very similar to the related compound Et 745 except for lack of the N¹²-methyl group.

The FABMS spectrum of Et 731 also showed lack of both the carbinolamine at C-21 and the N¹²-methyl group: The difference between the molecular ions observed in positive and negative ion FABMS for Et 731 was 2 Da, while Et's which have the carbinol amine at C-21 give an (M+H−H₂O)⁺ ion in positive and (M−H)— in negative FABMS, i.e., a difference of 16 Da (see Reference 4, cited below). These data along with new signals for the C-21 methylene (3.26 and 2.58 ppm) in the ¹H NMR spectrum support the above structure assignments. The FABMS/CID/MS spectrum of Et 731 showed intense fragment ions at m/z 204 and 190 (a and b in Scheme I), 14 Da less than those for Et 745, indicating lack of the N¹²-methyl group in the molecule. All the above data are consistent with the structure of Et 731 as N¹²-demethyl Et 745, depicted in Chart 1 (above). 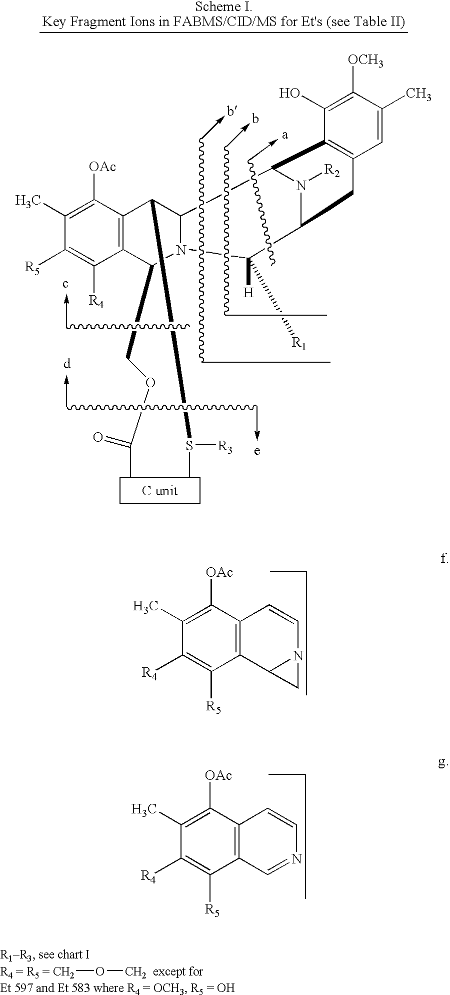
Structure of Et 745 B

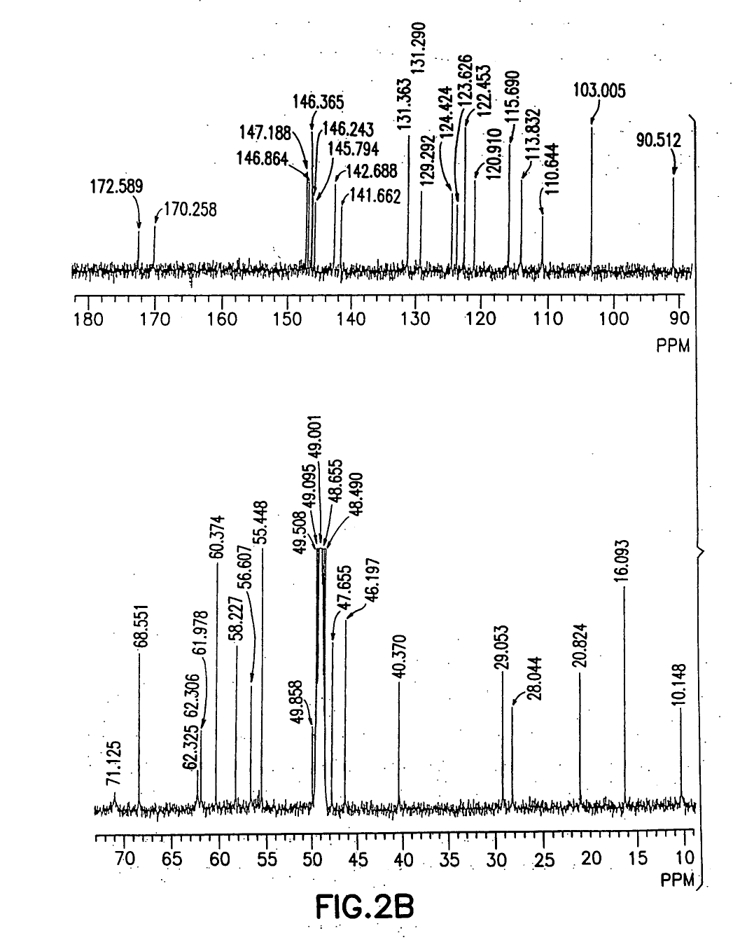

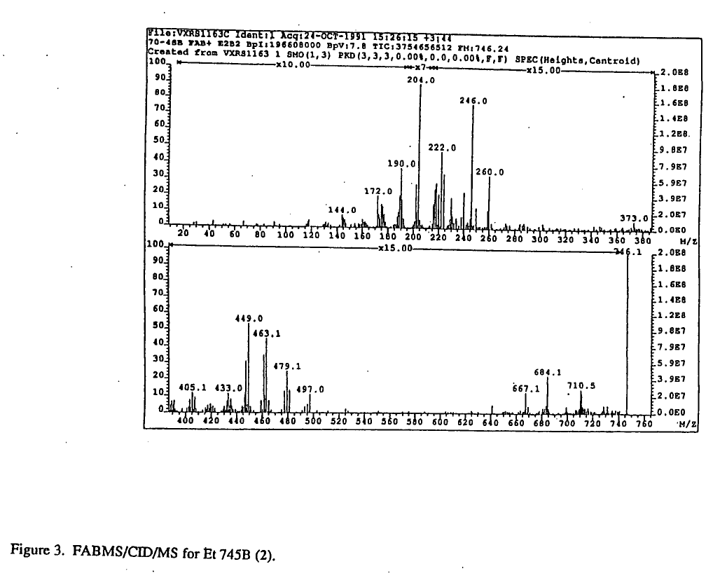

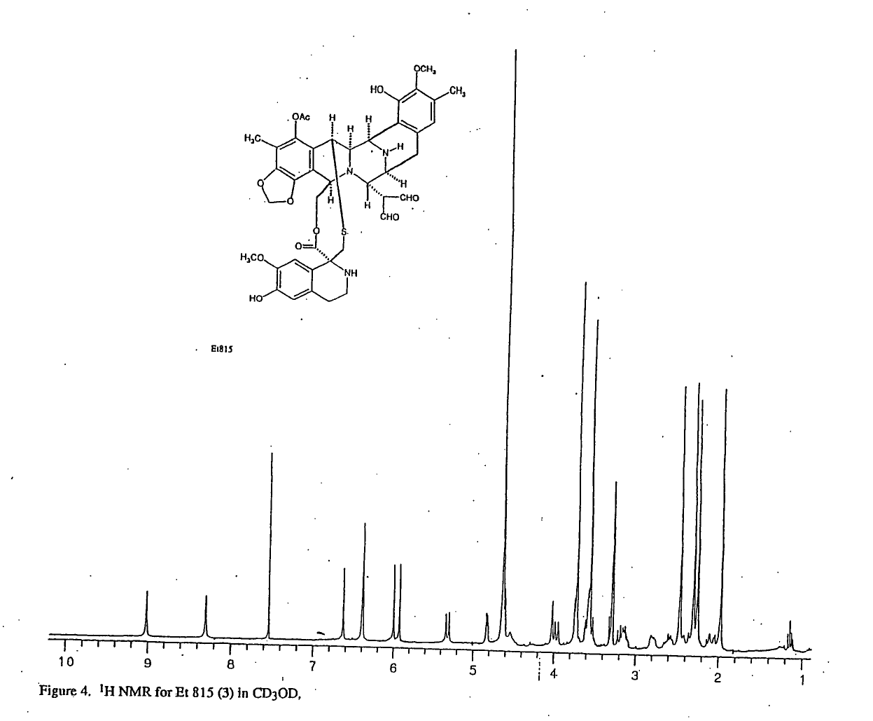

The positive ion HRFABMS spectrum of Et 745 B at m/z 746 (M+H−H₂O) agreed with the formula C₃₈H₄₀N₃O₁₁S for the dehydrated molecular ion. On the other hand, the methanol adduct ion at m/z 776 (M−H)⁻ was observed by negative ion FABMS when the sample was treated with methanol prior to measurement, with triethanolamine as matrix. These data indicated the presence of a reactive carbinolamine group in the molecule where small nucleophiles such as water or methanol can exchange, as observed for Et 743. See, for example, References 1 and 4, cited below. Thus, the hydrated molecular formula of Et 745 B must be C₃₈H₄₁N₃O₁₂S, which corresponds to the formula of Et 729 plus an oxygen. The ¹H and ¹³C NMR data for Et 745 B showed a pattern similar to that of Et 759, a sulfoxide derivative of Et 743, except for a lack of the N¹²-methyl group (see FIG. 2). FABMS/CID/MS data for Et 731 (see FIG. 3) showed m/z 190 and 204 for fragment ions a and b from unit A (Scheme I) and an ion at m/z 240 for fragment e from unit C. Although fragments a and b for Et 731 were the same as those for Et 729, fragment e at m/z 240 in Et 731 was 16 Da higher than that of Et 729. Since ¹H NMR signals for unit C of Et 731 were very similar to those of Et 729, the oxidation pattern on the tetrahydroisoquinoline rings in unit C of Et 731 is believed to be the same as that of Et 729. Thus the extra oxygen in unit C must be located on the sulfur atom, assigning the structure of Et 731 as the sulfoxide analog of Et 729.

Structure of Et 815

This structure was determined to be the 21-malonaldehyde derivative of Et 745. The molecular formula, C₄₂H₄₅N₃O₁₂S, was indicated by positive HRFABMS on the M+H ion at m/z 816 and negative ion FABMS data (m/z 814, M=H). Subtraction of the molecular formula for Et 745 (C₃₉H₄₃N₃O₁₀S) from the above formula gives a difference of C₃H₂O₂ which corresponds to the formula of a malonaldehyde substituent. In the ¹H NMR spectrum recorded in CD₃OD (see FIG. 4) two singlets for the aldehydes appeared at δ 9.03 and 8.28 but the proton α to the carbonyls was not observed, probably due to exchange of the α-proton by deuterium in CD₃OD. However, the ¹H NMR spectrum measured in acetone-d⁶ showed multiple resonances for each aldehyde proton, probably due to slow exchange of conformers. The HMBC spectrum recorded in acetone-d⁸ showed strong connectivity between H-21 and the aldehyde carbons and between the aldehyde protons and a carbon resonating at δ 57.7 ppm which is assignable to the α-carbon of the malonyl unit. It is interesting to note that strong correlations were observed in the HMBC spectrum between the aldehyde protons and a small carbon signal resonating at δ 115 ppm (see Scheme II). This can be assigned as an sp² α-carbon in the enol form.

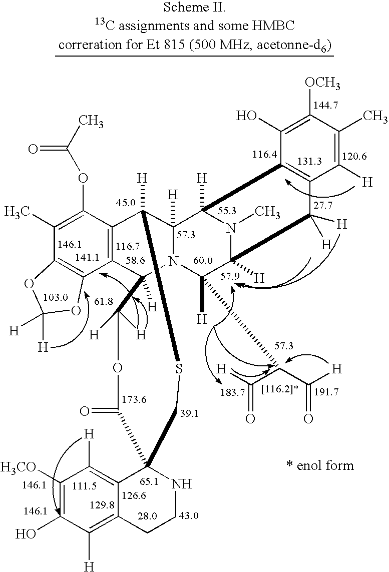

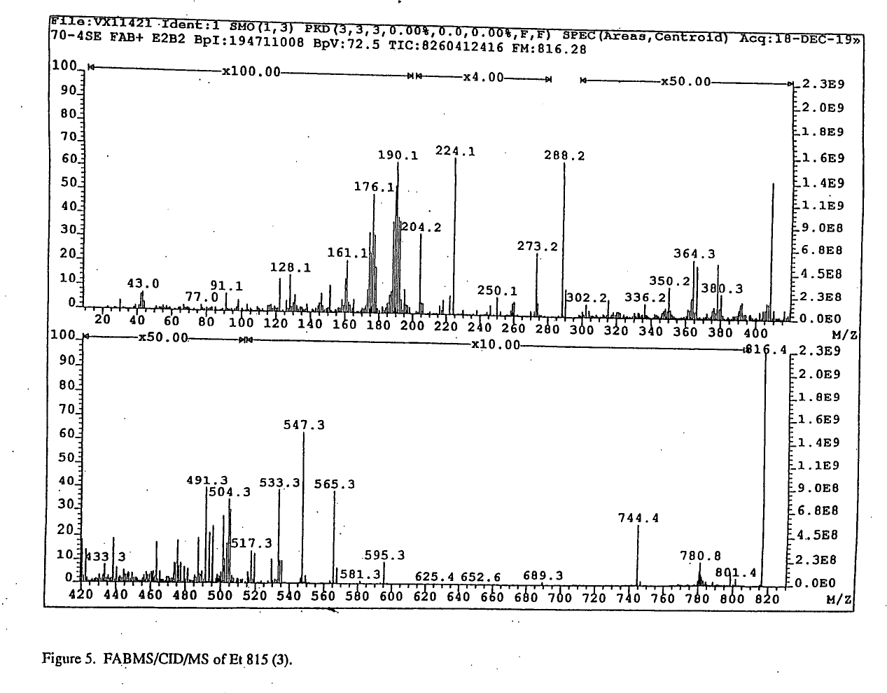

A FABMS/CID/MS spectrum for the molecular ion of Et 815 (see FIG. 5) showed fragments consistent with the above assignments; the ions b-d which contain the malonaldehyde group were shifted by 70 mu, whereas strong ions for a at m/z 224 where observed at the same masses as those of Et 745. Weak ions g and f for unit B at m/z 260 and 248, respectively, were also observed unchanged. These data indicated the presence of the malonaldehyde unit a C-21.

Structure of Et 808

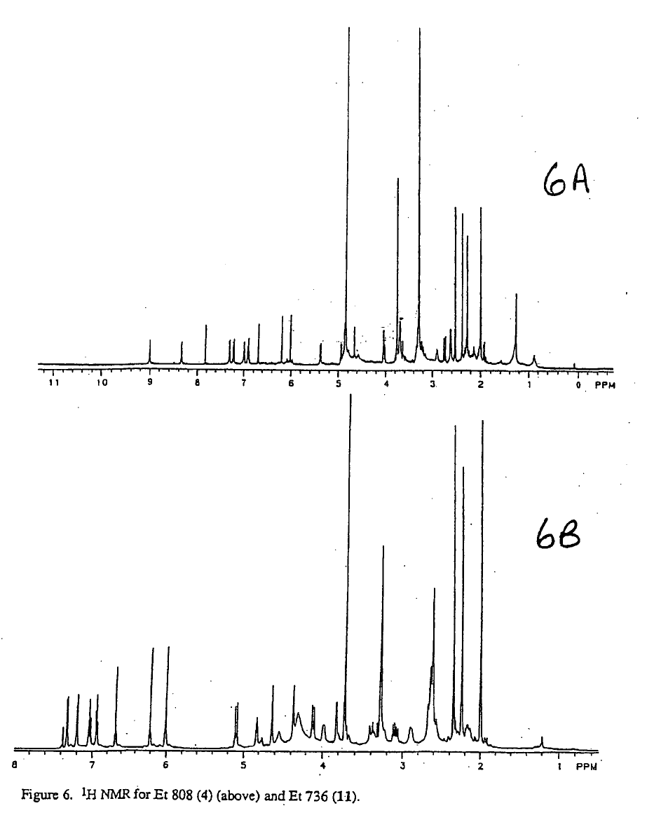

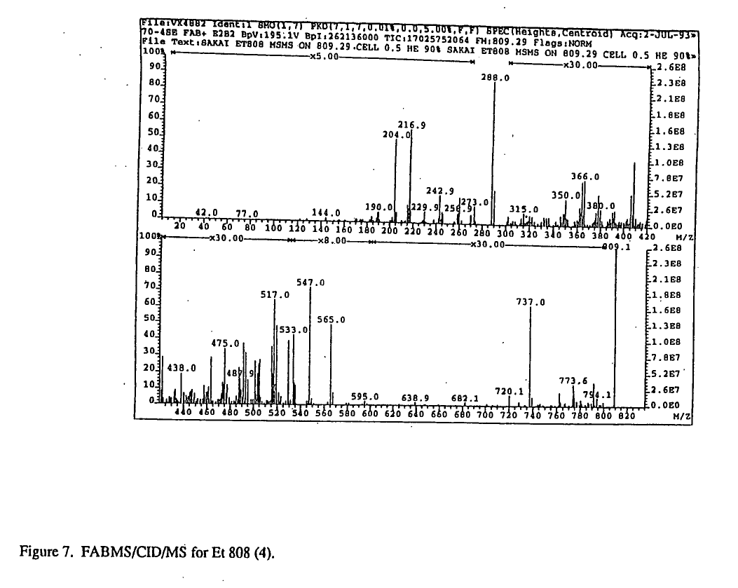

The ¹H NMR spectrum of Et 808 is very similar to that of Et 736 except for the appearance of two aldehyde protons at 9.02 and 8.36 ppm in Et 808 (see FIG. 6). The molecular formula C₄₂H₄₄N₄O₁₀S, assigned from positive ion HRFABMS data on the molecular ion (M+H)⁺ at m/z 809, is C₃H₄O₂ larger than that for M−H₂O of Et 736, which corresponds to a malonaldehyde group, assigning the structure of Et 808 to be the C-21 malonaldehyde analog of Et 736 (C-21 hydroxyl). FABMS/CID/MS data on Et 808(see FIG. 7) showing a fragmentation pattern similar to that of Et 815 (see Table II below) supported these structure assignments.

Structure of Et 596

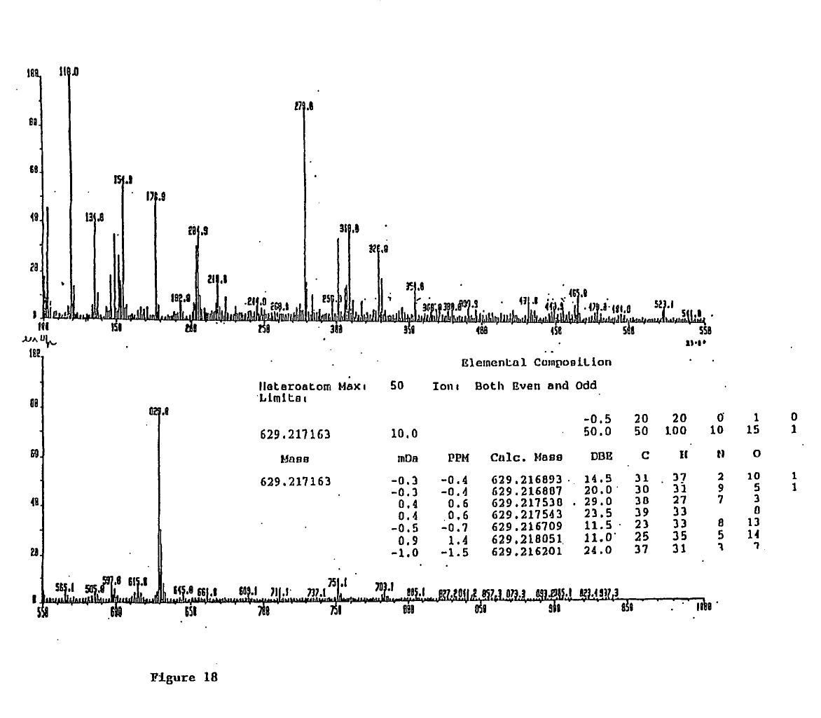

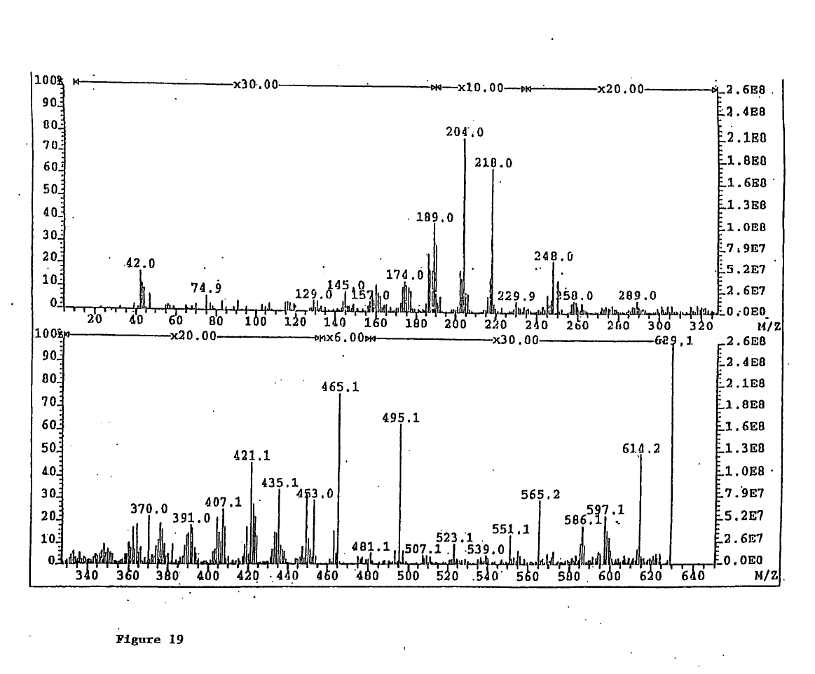

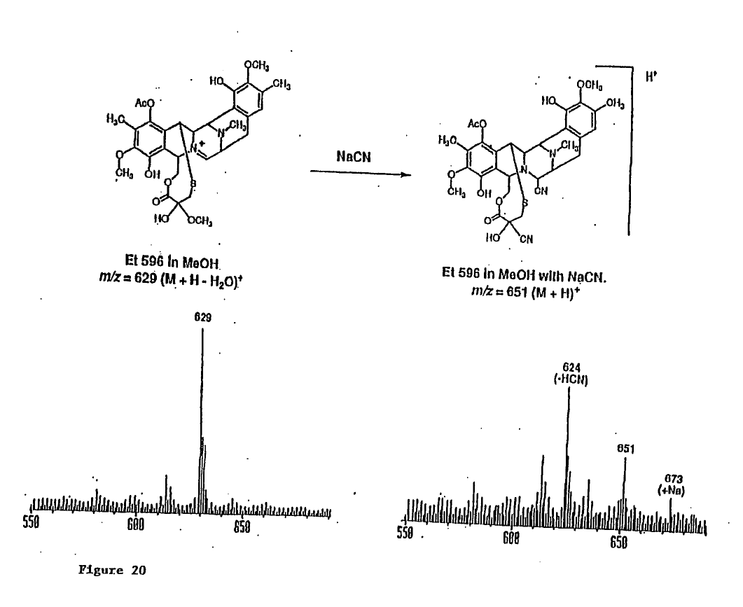

Fraction RS 2-12-6 (Example B-III, see below) was separated by HPLC (MeOH-0.04 M NaCl, 3:1) to afforded a fraction (0.5 mg) containing mainly Et 596. The structure of Et 596, was elucidated by FABMS data alone, due to the minute amount of Et 596 in the fraction. The molecular ion of Et 596 appeared at m/z 629 as a methanol adduct (FIG. 18). HRFABMS on this ion for Et 596 at m/z 629.2171 coincided with the formula of C₃₁H₃₇N₂O₁₀S suggesting the formula of Et 596 to be C₃₀H₃₂N₂O₉S. This molecular formula corresponds to that of Et 594 but with two more hydrogen atoms in Et 596. Along with this information, the electrophilic nature of this compound, as indicated by facile methanol adduct formation (similar to Et 594), suggested a presence of an α-keto C-unit in the molecule. The FABMS/CID/MS data (FIG. 19) indicated that the A and B units of Et 596 are the same as those of Et 597 (see below). Ions a and b for the A unit at m/z 204 and 218, respectively, remained unchanged (see Scheme II). On the other hand the ions from the B-unit and the A-B unit, namely f, g, and c, and d, respectively, are shifted by 2 mu as in the case of Et 597, indicating additional hydrogen atoms are located in the B-unit (see Scheme II). Addition of excess sodium cyanide in a methanol solution of Et 596, followed by FABMS measurement showed formation of mono- and di-cyano adducts which is indicated by new ions at m/z 624 and 651, respectively (FIG. 20). This result confirmed the presence of the carbinol amine group at C-21 and the α-keto functionality in the C-unit. From all of these data, the structure of Et 596 was assigned as depicted.

Crude Et 596 (as a single major peak by FABMS in the m/z 500-800 region, see FIG. 18) exhibited antimicrobial activity against B. subtilis at 0.3 μg/disc (MIC).

Structure of Et 597

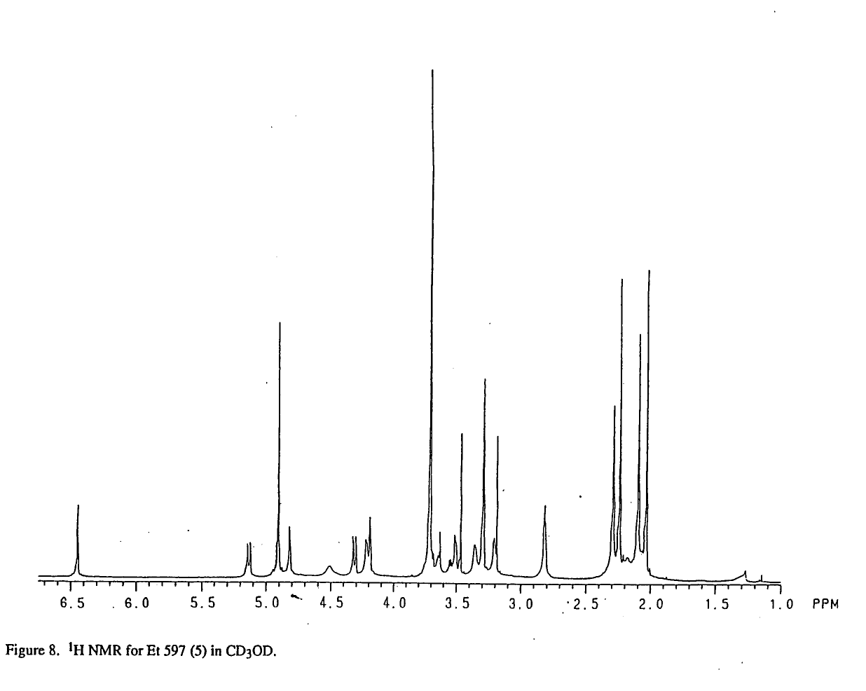

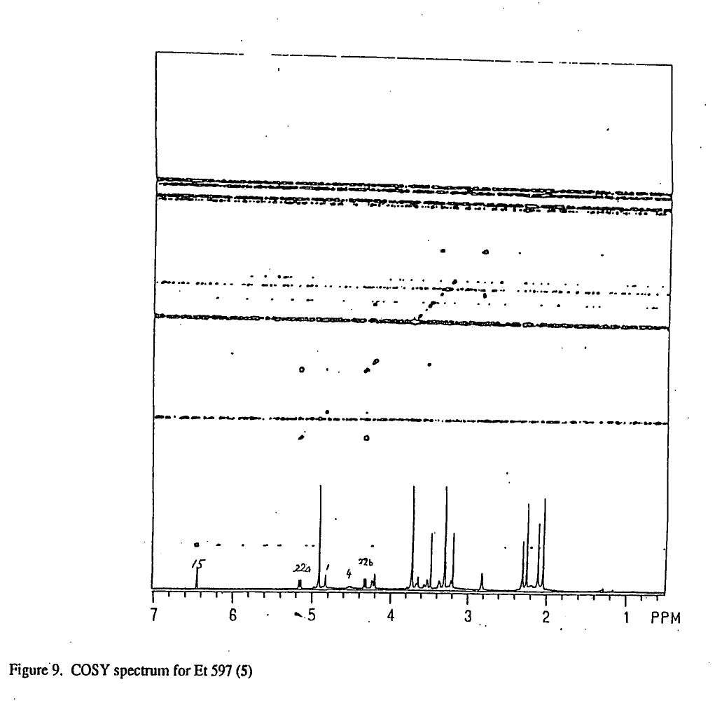

The ¹H NMR spectrum of Et 597 (see FIG. 8) appeared much simpler in the low field region than those of other Et's, containing only one aromatic proton and lacking a methylenedioxy unit. Also, the X—CH₂—CH₂—Y system in the region between 2.5-3.4 ppm typical of the tetrahydroisoquinoline unit C in Et 743-type compounds was missing. However, the ¹H NMR signals assigned by COSY (see FIG. 9), HMQC, and HMBC (see Table I, below) for the aliphatic portion of the A-B units of Et 597 had chemical shifts and coupling constants very similar to those of Et 743. Two aromatic methoxyl groups were also present in the ¹H NMR spectrum of Et 597 despite the lack of unit C. These data indicated major differences between the structures of Et's 597 and 743, which can be attributed to the unit C.

TABLE 1
¹H and ¹³C NMR Data for Et's 743 in CD₃OD-CDCl₃ (3:1), 597, 583, and 594 in CD₃OD
Chemical shift (δ), multiplicity^{a} (J in Hz).

| | Et 743 | | | Et 597 | | Et 583 | | Et 594 | |
|---|---|---|---|---|---|---|---|---|---|
| # atoms^{b} | ¹³C | ¹H | # atoms | ¹³C | ¹H | ¹³C | ¹H | ¹³C | ¹H |
| 1 | 56.3, d | 4.78, br s | 1 | 57.2, d | 4.82, br s | 58.2, d | 4.73 brs | 57.0, d | 4.78, brs |
| 3 | 58.8, d | 3.72^{c} | 2 | 58.9 d | 3.51 br d(3.5) | 58.5, d | 3.47 brd(5.0) | 59.5, d, | 3.58 d(4.5) |
| 4 | 42.7, d | 4.58, br s | 3 | 43.1, d | 4.51, br s | 48.4, d | 4.50 brs | 42.5 | 4.45 |
| 5 | 142.2, s | | 4 | 140.3, s | | | | | |
| 6 | 113.9, s | | 5 | 124.3, s | | | | | |
| 7 | 146.5, s^{d} | | 6 | 146.5, s^{d} | | | | | |
| 8 | 141.9, s | | 7 | 144.7, s | | | | | |
| 9 | 116.0, s | | 8 | 122.1 s | | | | | |
| 10 | 122.0, s | | 9 | 115.6, s | | | | | |
| 11 | 55.6, d | 4.40, br d(3.5) | 10 | 56.0, d | 4.22 brd, (4.0) | 48.8, d | 4.28 d(4.5) | 56.5, d | 4.21 m |
| 13 | 54.0, d | 3.52, br s | 13 | 54.1, d | 3.37, brm | 4.72, d | 3.63 brdd(8.5, | 55.1 | 3.38 m |
| | | | | | | | 2.5) | | |
| 14 | 24.5, t | 2,91, 2H, br d(4.5) | 14 | 25.6, t | 2.82, d, (5.0) | 28.1, t | 2.98 dd(17.5, 9.5) | 24.9 | 2.81 dd(17.0, 9.0) |
| | | | | | | | 3.07 d(17.5) | | 2.69 d(17.0) |
| 15 | 120.9, d | 6.55, s | 15 | 121.2, d | 6.45, s | 122.1, d | 6.49 s | 121.7 d | 6.43 s |
| 16 | 131.2, s | | 16 | 130.9, s | | | | | |
| 17 | 145.1, s | | 17 | 145.7, s | | | | | |
| 18 | 149.8, s | | 18 | 150.3, s | | | | | |
| 19 | 119.2, s | | 19 | 120.3, s | | | | | |
| 20 | 131.5, s | | 20 | 132.1, s | | | | | |
| 21 | 92.1, d | 4.26, d(3.0) | 21 | 93.1, d | 4.19, d(3.0) | 91.5, d | 4.15 d(2.5) | 91.7, d | 4.21 m |
| 22 | 61.2, t | 5.14, d(11.0) | 22 | 61.4, t | 5.14, d(11.0) | 62.1 | 5.14 d(11.0) | 62.3, t | 5.16 d(11.5) |
| | | 4.09, dd(11.0, 2.0) | | | 4.31, dd(2.0, | | 4.32 dd(11.0, 2.0) | | 4.08 dd(11.5, 2.5) |
| | | | | | 11.0) | | | | |
| OCH₂O | 103.1, t | 6.07, d(1.0) | | | | | | 103.6 t | 6.11 d(1.0) |
| | | 5.98, d(1.0) | | | | | | | 6.00 d(1.0) |
| 1′ | 65.3, s | | 2′ | 54.3, d | 3.22, brm | 54.9, d | 3.22 brm | | |
| 3′ | 40.3, t | 3.13, dt(11.0, 4.0) | | | | | | | |
| | | 2.77 ddd(3.5, 5.5, 11.0) | | | | | | | |
| 4′ | 28.6, t | 2.60, ddd(5.5, 10.5, | | | | | | | |
| | | 16.0) | | | | | | | |
| | | 2.42, ddd(3.5, 3.5, 16.0) | | | | | | | |
| 5′ | 115.6, d | 6.38, s | | | | | | | |
| 6′ | 146.4, s^{d} | | | | | | | | |
| 7′ | 146.4, s^{d} | | | | | | | | |
| 8′ | 111.3, d | 6.42, br s | | | | | | | |
| 9′ | 125.4, s | | | | | | | | |
| 10′ | 128.8, s | | | | | | | | |
| 11′ | 173.1, s | | 1′ | 174.8, s | | | | 100.5, s | |
| 12′ | 43.1, t | 2.38, br d(15.5) | 3′ | 35.4, t | 2.2 | 35.5, t | 2.2 | 38.7, t | 1.84 d(15.0) |
| | | 2.05^{c} | | | | | | | |
| 5 C═O | 169.8, s | | 5 C═C | 167.5, s | | | | | |
| 5 OAc | 20.5, q | 2.29, s | 5 OAc | 20.8, q | 2.29, s | 21.2 q | 2.29 s | 20.4, q | 2.31 s |
| 6 CH₃ | 9.9, q | 2.01, s | 6 CH₃ | 10.1, q | 2.04, s | 10.4 q | 2.03 s | 9.7, q | 1.99 s |
| 7 CH₃ | | | 7 CH₃ | 61.1, q | 3.71, s | 61.4 q | 3.70 s | 60.2 q | 3.70 s |
| 16 CH₃ | 16.1, q | 2.28, s | 16 CH₃ | 15.9, q | 2.24, s | 15.9, q | 2.23 s | 16.1, q | 2.22 s |
| 17 OCH₃ | 60.2, q | 3.72, s | 17 OCH₃ | 60.2, q | 3.72, s | 60.3, q | 3.72, s | 60.3 q | |
| 7′ OCH₃ | 55.7, q | 3.58, s | | | | | | | |
| 12 NCH₃ | 41.1, q | 2.23, s | 12 NCH₃ | 41.2, q | 2.01 s | | | 40.8, q | 2.06 s |

^{a}s = singlet, d = doublet, t = triplet, q = quartet, br = broad.
^{b}Proton assignments are based on COSY and homonuclear decoupling experiments; carbon multiplicities were determined based on APT and DEPT and HMQC data.
^{c}Signals overlap the methyl singlet.
^{d}Assignments are interchangeable. ^{f}Carbon resources were observed through proton resonances by HMQC experiment due to the limited amount of samples available.

TABLE II
FABMS Data of Ecteinascidines (See Scheme II)
A. C-21-carbinolamine derivatives

| | | | | fragment (MS/MS or HRFABMS) | | |
|---|---|---|---|---|---|---|
| compound | formula | M + H-H₂O (obs) | M − H | a | b | c |
| Et 743^{a} | C₃₉H₄₃N₃O₁₁S | C₃₉H₄₂N₃O₁₀S | C₃₉H₄₂N₃O₁₁S | C₁₂H₁₄NO₂ | C₁₃H₁₆NO₂ | C₂₆H₂₇N₂O₆ |
| | | 744.2591 Δ 5.7 | 760.2514 Δ 2.6 | 204.1025 | 218.1174 | 463.1862 |
| Et 729^{a} | C₃₈H₄₁N₃O₁₁S | C₃₈H₄₀N₃O₁₀S | C₃₈H₄₀N₃O₁₁S | C₁₁H₁₂NO₂ | C₁₂H₁₄NO₂ | C₂₅H₂₅N₂O₆ |
| | | 730.2493 Δ −5.0 | 746.2376 Δ 0.8 | 190 | 204 | 449 |
| Et 759C | C₃₉H₄₃N₃O₁₂S | C₃₉H₄₂N₃O₁₁S | C₃₉H₄₂N₃O₁₂S | 204 | 218 | 479 |
| | | 760.2540 Δ 0.6 | | | | |
| Et 759B | C₃₉H₄₃N₃O₁₂S | C₃₉H₄₂N₃O₁₁S | C₃₉H₄₂N₃O₁₂S | 204 | 218 | 463 |
| | | 760.2550 Δ −1.8 | 776.2446 Δ 4.3 | | | |
| Et 745B | C₃₈H₄₁N₃O₁₂S | C₃₈H₄₀N₃O₁₁S | 776^{b} | 190 | 204 | 449 |
| | | 746.2398 Δ −1.4 | | | | |
| Et 736 | C₄₀H₄₂N₄O₉S | C₄₀H₄₁N₄O₈S | C₄₀H₄₁N₄O₉S | 204 | 218 | 463 |
| | | 737.2655 Δ −1.8 | 753.2588 Δ −0.5 | | | |
| Et 722 | C₃₉H₄₀N₄O₉S | C₃₉H₃₉N₄O₈S | C₃₉H₃₉N₄O₉S | 190 | 204 | 449 |
| | | 723.2496 Δ −0.7 | 739.2433 Δ 0.7 | | | |
| Et 597 | C₃₀H₃₇N₃O₉S | C₃₀H₃₆N₃O₈S | NO | 204 | 218 | 465 |
| | | 598.2219 Δ 0.4 | | | | |
| Et 583 | C₂₉H₃₅N₃O₉S | C₂₉H₃₄N₃O₈S | NO | 190 | 204 | 451 |
| | | 584.2054 Δ 1.2 | | | | |
| Et 594^{c} | C₃₀H₃₂N₂O₁₀S | C₃₀H₃₁N₂O₉S | NO | 204 | 218 | 463 |
| | | 595.1716 Δ 3.4 | | | | |

| | fragment (MS/MS or HRFABMS) | | | |
|---|---|---|---|---|
| compound | d | e | f | g |
| Et 743^{a} | C₂₇H₂₉N₂O₇ | C₁₁H₁₄NO₂S | C₁₄H₁₄NO₄ | C₁₃H₁₂NO₄ |
| | 493.1980 | 224 | 260 | 246 |
| Et 729^{a} | C₂₆H₂₇N₂O₇ | 224 | 260 | 246 |
| | 479 | | | |
| Et 759C | 509 | C₁₁H₁₄NO₃S | 260 | 246 |
| | | 224 | | |
| Et 759B | 493 | C₁₁H₁₄NO₃S | NO^{d} | 246 |
| | | 240 | | |
| Et 745B | 479 | 240 | 260 | 246 |
| Et 736 | 493 | C₁₃H₁₁N₂OS | 260 | 246 |
| | | 243.0593 | | |
| Et 722 | 479 | 243 | 260 | 246 |
| Et 597 | 495 | NO | 262 (s)^{e} | 248 |
| Et 583 | 481 | NO | 262 (s) | 248 |
| Et 594^{c} | 493 | NO | NO | NO |

B. C-21 Substituted by other than OH

| compound | formula | M + H (obs) | M − H | a | b | c | d | e | f | g |
|---|---|---|---|---|---|---|---|---|---|---|
| Et 745^{a} | C₃₉H₄₃N₃O₁₀S | NO | 204 | 218 | 463 | 493 | 224 | 260 | 246 | |
| | | 732.2606 | | | | | | | | |
| | | Δ −1.5 | | | | | | | | |
| Et 731 | C₃₈H₄₁N₃O₁₀S | C₃₈H₄₂N₃O₁₀S | C₃₈H₄₀N₃O₁₀S | 190 | 204 | 449 | 481 | 224 | 260 | NO |
| | | 732.2606 | 730.2422 Δ 1.2 | | | | | | | |
| | | Δ −1.5 | | | | | | | | |
| Et 815 | C₄₂H₄₅N₃O₁₂S | C₄₂H₄₆N₃O₁₂S | 814 | 204 | 288 | 533 | 565 (2H) | 224 | 260 (s) | 246 (s) |
| | | 816.2788 Δ 1.4 | | | | | | | | |
| Et 808 | C₄₃H₄₄N₄O₁₀S | C₄₃H₄₅N₄O₁₀S | | 204 | 288 | 533 | 565 | 243 | 260 | 246 |
| | | 809.2851 Δ 0.5 | | | | | | | | |
| Et 770^{a} | C₄₀H₄₂N₄O₁₀S | C₄₀H₄₃N₄O₁₀S | | 204 | 244 | 488 | 502 | 224 | NO | NO |
| | | 771.2704 | | | | | | | | |
| | | Δ −0.4 | | | | | | | | |

^{a}Data taken from Ref 4.
^{b}Methanol adduct.
^{c}MS/MS on m/z 627 (M + MeOH).
^{d}NO = not observed.
^{e}(s) = small peak.

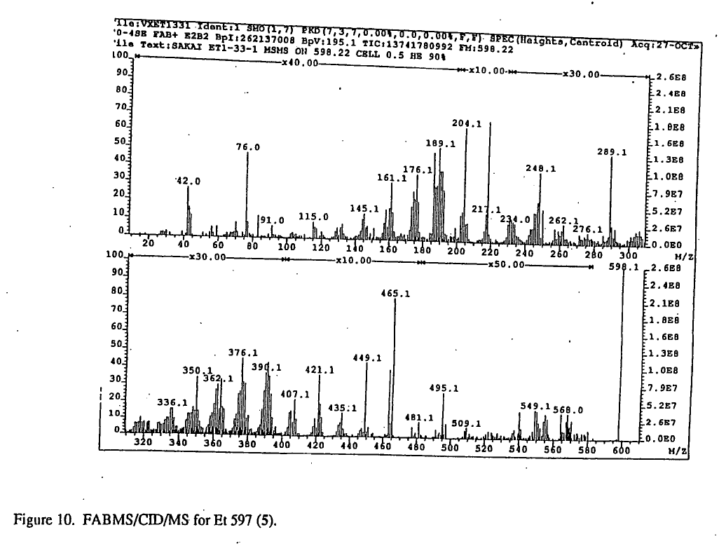

The positive ion HRFABMS data on m/z 598 of Et 597 agreed with the formula C₃₀H₃₆N₃O₈S (M+H−H₂O). Unfortunately, negative ion FABMS did not give an M−H peak due to lack of sensitivity. The actual molecular formula of Et 597 was assigned to be C₃₀H₃₇N₃O₉S, since the presence of the C-21 carbinolamine group was indicated by ¹H and ¹³C NMR signals (δ 4.19 and 93.1 ppm, respectively). FABMS/CID/MS data for Et 597 (see FIG. 10) and Et 743 on M+H−H₂O ions were compared. Both showed intense fragments a and b at m/z 218 from unit A of Et 597 while fragments c and d were at m/z 465 and 495 and product ions at m/z 262 and 248 assignable to fragments f and g from unit B of 6 are at 2 Da higher mass than those of Et 743 (see Scheme I and Table II). These data suggested that the unit A of Et 597 has the same structure as in Et 743, while unit B of Et 597 contains two more hydrogens than in Et 743. These data and the above ¹H NMR data, which showed lack of a methylenedioxy group and an additional methoxyl group, can be accounted for if the methylenedioxy group in unit B is replaced by methoxy and hydroxyl groups.

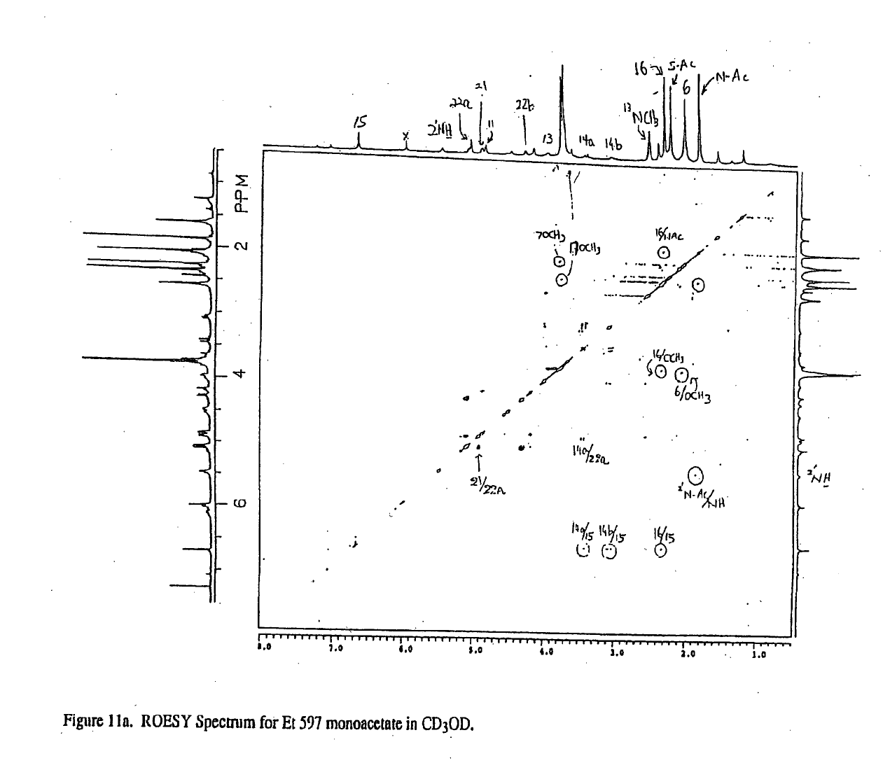

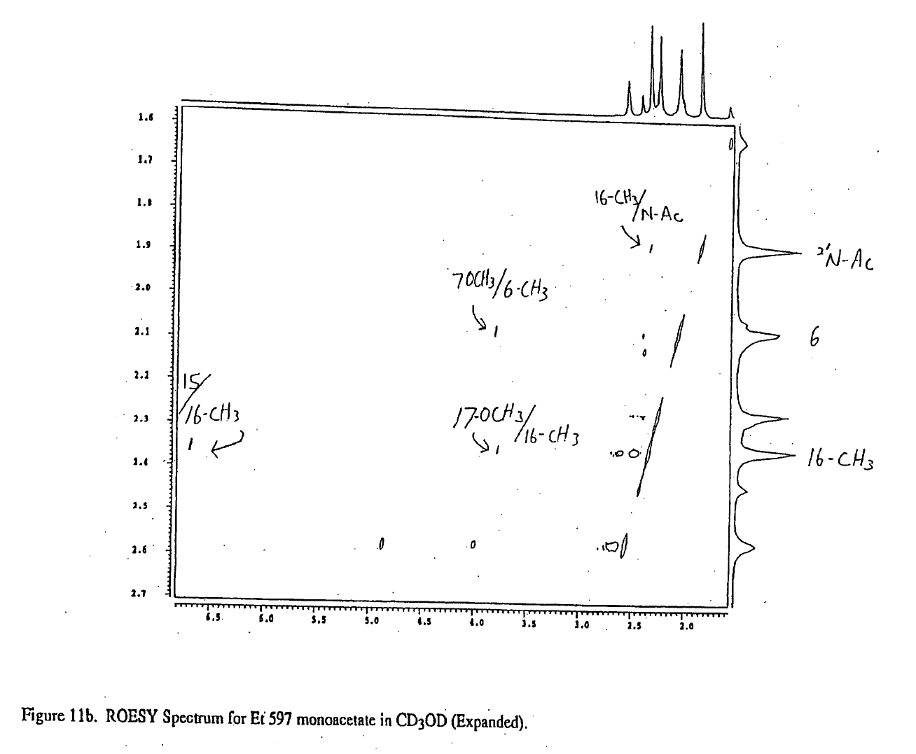

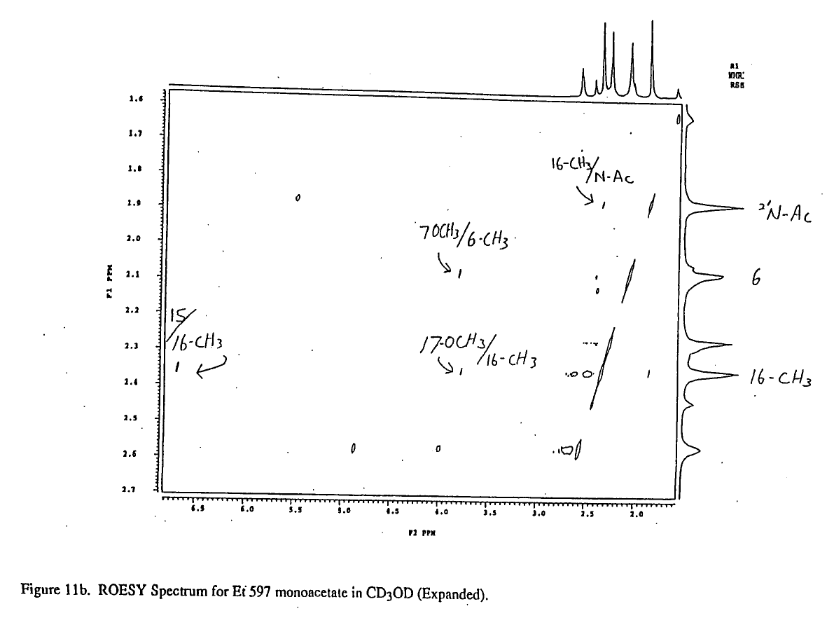

The position of the methoxy group (on C-7) was confirmed by ROESY NMR data for monoacetyl Et 597 (500 MHz, CDCl₃, FIG. 11), prepared by treating Et 597 with Ac₂O and TEA, which showed ROESY cross peaks between two benzylic methyl groups and two methoxyl groups, indicating these groups are next to each other in both units A and B. The ROESY data also confirmed the relative stereochemistry of the A-B unit to be the same as that in Et 743, since all common correlations found in Et's were observed in the ROESY spectrum of Et 597 (see Scheme III).

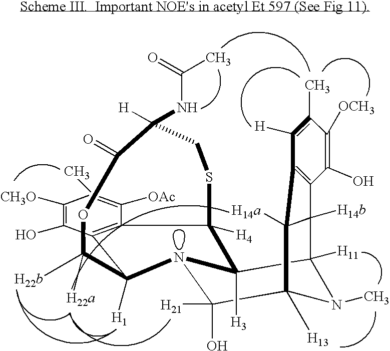

All the above data indicated the molecular formula for the A-B unit of Et 597 to be C₂₇H₃₁N₂O₇, the same as that of Et 743 plus two additional hydrogens in unit B. Thus, the rest of the molecule must be C₃H₆NOS, which accommodates two degrees of unsaturation.

Since the ¹³C NMR spectrum showed the presence of two ester carbonyl groups at δ 167.4 and 174.6 ppm, and the former was assigned to be the acetyl carbonyl in unit B by HMBC, the oxygen in the above formula was attributed to the remaining ester carbonyl which links unit C to unit B.

COSY and HMBC data for Et 597 showed that the spin system —CH—CH₂—O—CO—, which is commonly observed in the other Et's for C-1, C-22 and the ester carbonyl of unit C, is also present in this molecule. The HMQC data showed that a broad singlet observed at δ 3.22 ppm is correlated to a carbon resonating at δ 54.3 ppm, suggesting the presence of an amine. This proton shifted to δ 4.53 ppm on acetylation of Et 597 and was coupled to an exchangeable proton at δ 5.48 ppm, confirming the presence of the primary amino group. A sulfur attached to C-4 is suggested by the NMR data, since resonances for H-4 (δ 4.51 ppm) and C-4 (δ 43.1 ppm) are very similar to those of other Et's (c.f. Et 743, Table I). A methylene carbon resonating at δ 35.4 ppm and correlating to a very broad proton signal at δ 2.2 ppm by HMQC is assignable to a sulfide carbon. Unfortunately, no correlation spectra (COSY, HMBC) connected the sulfide methylene and a proton (or carbon) α to the ester carbonyl. However, these two groups must be connected to form a 10-membered sulfide-containing lactone, like all other Et's, to agree with the required level of unsaturation. Thus, the structure of Et 597 was assigned as depicted above in Chart I.

Absolute Stereochemistry of Et 597

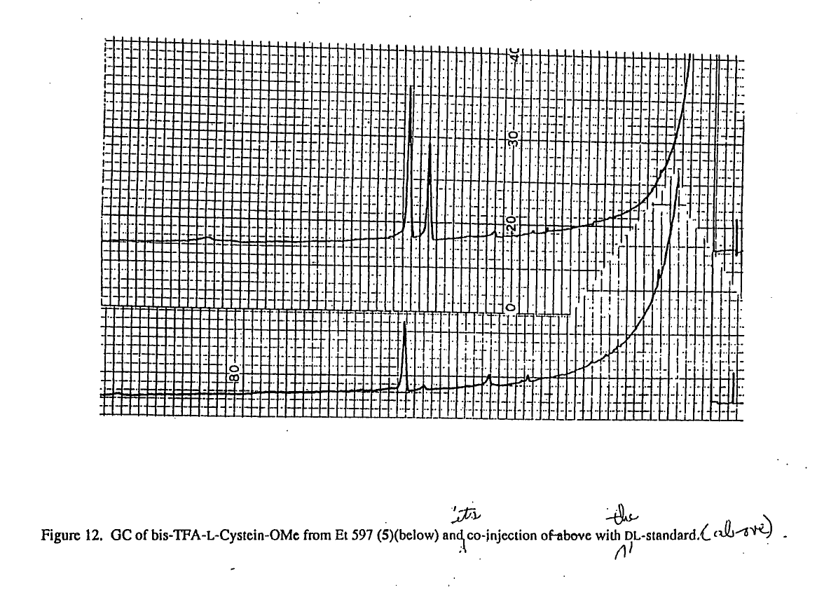

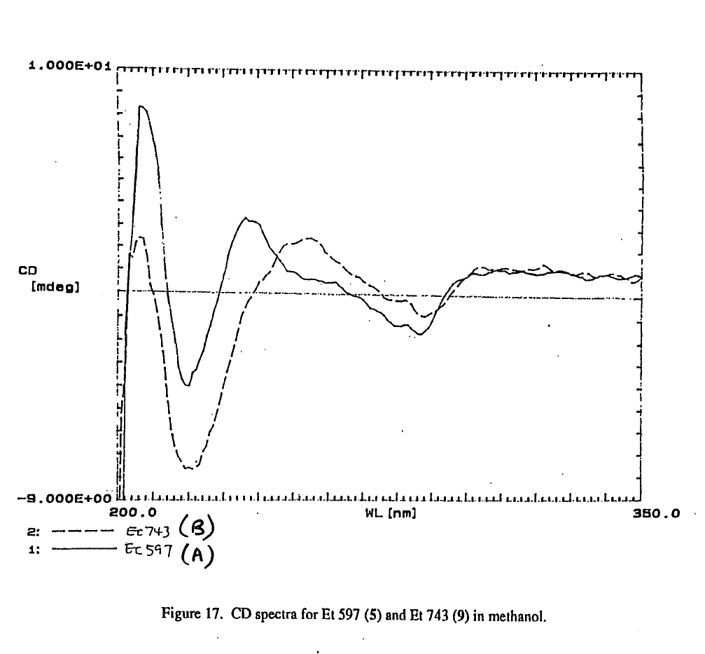

A ROESY NMR spectrum of the monoacetyl derivative of Et 597 showed an NOE between the amine proton and the methyl protons of the acetamide group of the C unit (see FIG. 11). An NOE between the acetyl methyl group and the methyl group at C-16 of unit A revealed that the relative stereochemistry of the secondary amine is as depicted in Chart I and Scheme III, in which the amide nitrogen must face toward the aromatic ring of the unit A. Treatment of Et 597 with HgCl₂ followed by NaBH₄ then methanolysis give a mixture containing cysteine methyl ester. This product was derivatized with trifluoroacetic anhydride (TFAA) and the TFA derivative was then analyzed by chiral GC and GC/MS. Injection of the derivatized sample with a D,L-mixture of TFA-Cys-OMe showed that the Cys in the derivatized sample coelutes with the L-isomer of the standard mixture (see FIG. 12). Thus, the absolute stereochemistry at C-2′ of Et 597 was determined to be R. Since the relative stereochemistry of the C unit and the AB unit was related by the above NOE experiment, and also the relative stereochemistry of the A-B unit of Et 597 was shown to be the same as that of Et 743, the stereochemistry of Et 597 is assigned as 1R, 2R, 3R, 4R, 11R, 13S, 21S, 2′R. CD data for Et 597 were very similar to those for Et 743 (see FIG. 17), indicating the absolute configuration of Et 743 is the same as that of Et 597.

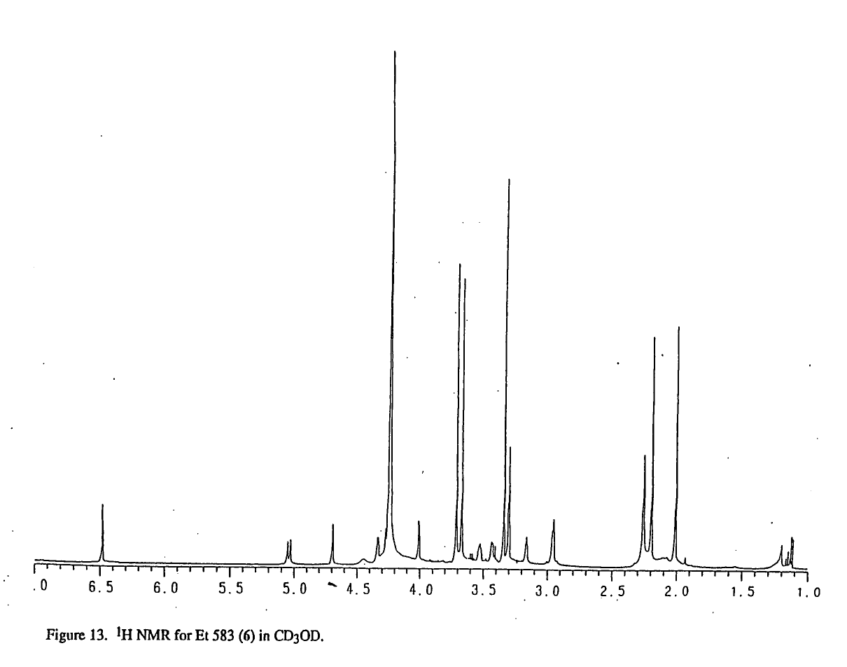

Ecteinascidin 583 was determined to be an N¹²-demethyl analog of Et 597. In the ¹H NMR spectrum (see FIG. 13) only three methyl groups are observed in the region of δ 2.0 to 2.5 ppm whereas four methyl signals appeared in the spectrum of Et 597. Positive ion FABMS data for Et 583 showed an M+H−H₂O peak at m/z 584. HRFABMS data on this ion agreed with the molecular formula C₂₉H₃₃N₃O₈S. Since the presence of a carbinolamine at C-21 was evident from the ¹H NMR resonance at δ 4.15 ppm, the actual (hydrated) molecular formula of Et 583 (with 21-hydroxyl) is assigned to be C₂₉H₃₅N₃O₉S, one CH₂ less than that of Et 597, corresponding to the difference mentioned above.

COSY and HMQC of Et 583 in Comparison to Other Et's

NMR data allowed assignment of all the protons and protonated carbons as in Table I in which C-11 and C-1 3 are shifted upfield compared to those carbons of Et 597 as a result of the β-effect at N-12, while ¹H NMR signals are shifted downfield. These shifts in the NMR are commonly observed between the N¹²-methyl and N¹²-demethyl analogs of Et's.

Ecteinascidin 594

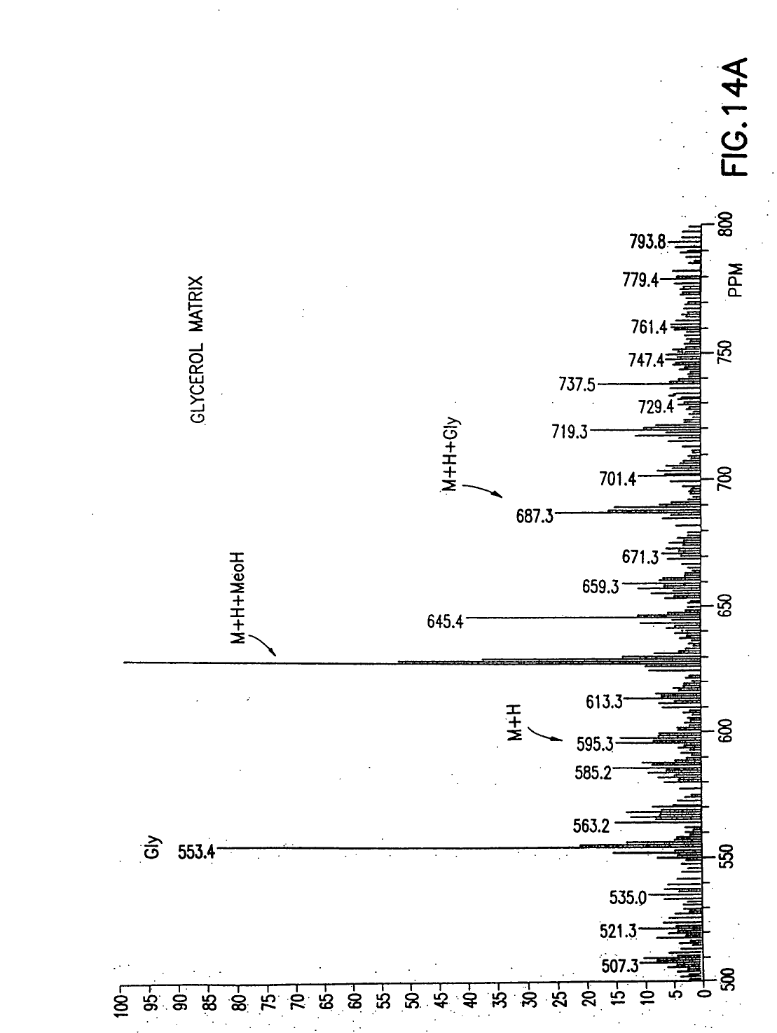

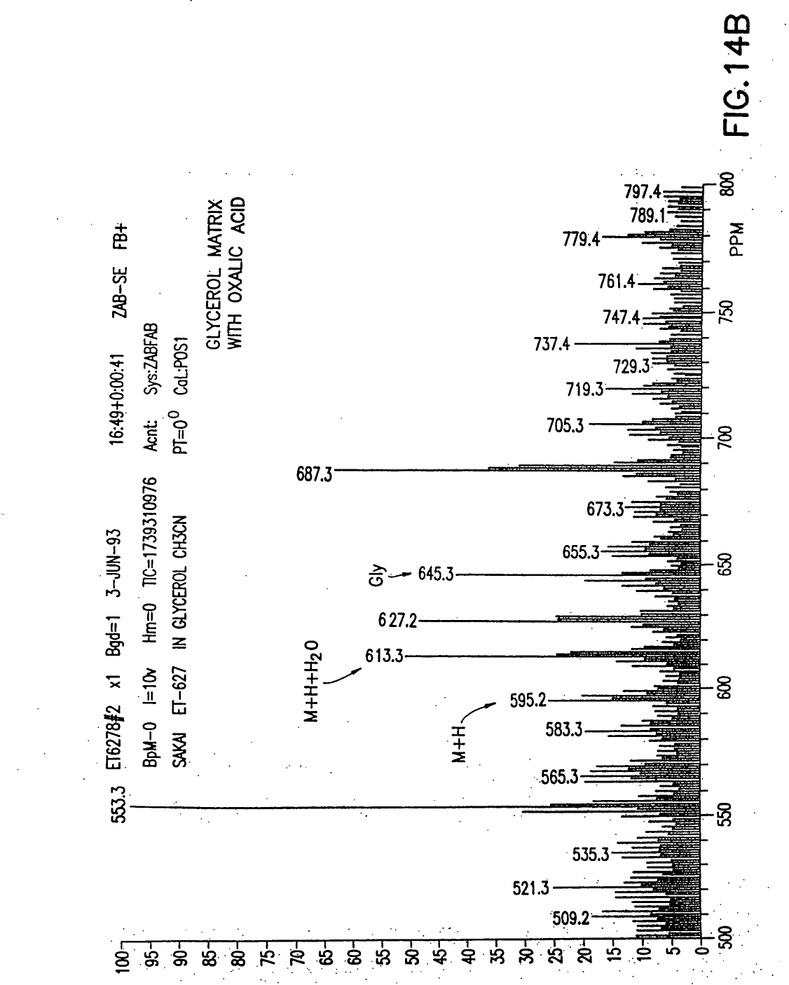

Et 594 was obtained as a methanol adduct, giving a protonated molecular ion (M+H) at m/z 627 in magic bullet (MB) matrix (containing 10% methanol). HRFABMS data for the methanol adduct (m/z 627.2020) agreed with the formula C₃₁H₃₅N₂O₁₀S (M+H+MeOH−H₂O). The molecular ion of Et 594 was observed in FABMS spectra in a glycerol matrix when a trace amount of oxalic acid was added. The FABMS spectra in glycerol matrix alone gave only the M+H+MeOH ion at m/z 627; however, peaks at m/z 596, 613 and 687 were observed when a small amount of oxalic acid and water was added (see FIG. 14). HRFABMS of each of the above peaks agreed with formulas for [M+H]⁺ (C₃₀H₃₁N₂O₉S, 595.1750, Δ 3.4 mmu), [M+H+H₂O ]⁺(C₃₀H₃₃N₂O₁₀S, 613.1827, Δ 2.9 mmu, and [M+H+glycerol]⁺ (C₃₃H₃₉N₂O₁₂S, 687.2205, Δ 1.8 mmu), respectively.

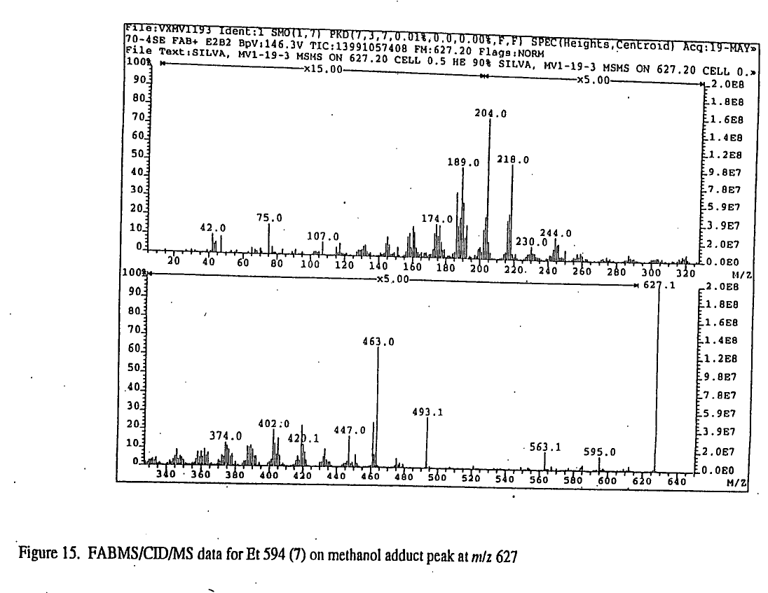

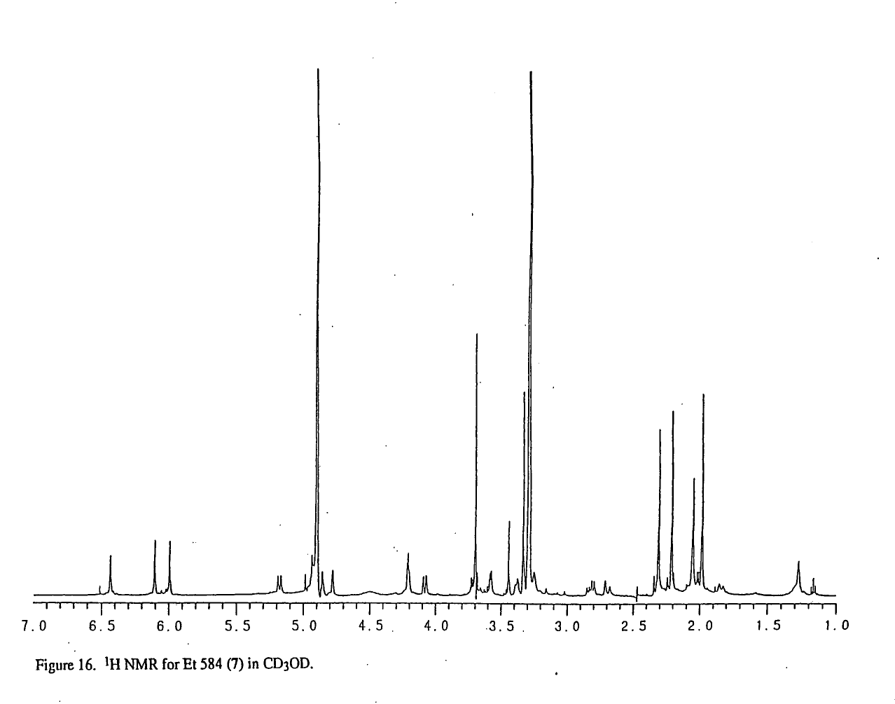

In the COSY data a proton resonance assignable to H-21 appeared at δ 4.21 ppm, indicating the presence of a carbinolamine group in Et 594. From these data, the molecular formula of Et 594 (C-21 hydroxyl) was established as C₃₀H₃₂N₂O₁₀S. FABMS/CID/MS spectra of the methanol adduct (m/z 627, see FIG. 15) gave product ions at m/z 204, 218, 463 and 493, which.correspond to the fragments a-d (see Scheme I and Table. II), common in Et 743, and suggest the unit A-B of Et 594. is the same as that of Et 743. A ¹H NMR spectrum of Et 594 recorded in CD₃OD (see FIG. 16) showed only one aromatic singlet, for H-15 at δ 6.43 ppm, which showed a COSY cross peak to the methyl resonance (16-CH₃), and two protons for the methylenedioxy at δ 6. 10 and 6.00 ppm. Other resonances were very similar to those of Et 597, except that the signal for CHNH₂ in Et 597 which appeared at δ 3.22 ppm was missing for Et 729, suggesting the A-B unit of Et 729 and Et 597 is the same except for the methylenedioxy unit. Thus the structure of Et 594 was assigned as including a 2′-oxo group instead of a 2′-amino in the C unit and as having a methylenedioxy group in the B unit as depicted in Chart I.

Bioactivities of the New Et's.

All the above new Et's discussed herein exhibited strong cytotoxicity against several tumor cell lines and a normal cell line. The results are summarized below in Table III, below.

TABLE III
Cytotoxicities^{a} Antimetabolism^{b}, Enzyme Inhibition^{c}, and Antimicrobial Activity^{d} of of Et's.

| | | | | | | | | | | | | B. s.^{d} |
|---|---|---|---|---|---|---|---|---|---|---|---|---|
| | IC₅₀ (ng/mL) | | | | | | IC₅₀ (μg/mL) | | | | | MIC |
| | L1210^{a} | P388^{a} | A549^{a} | HT29^{a} | MEL28^{a} | CV-1^{a} | Prot.^{b} | DNA^{b} | RNA^{b} | DNAp^{c} | RNAp^{c} | μg/disc |
| Et 743 | 5 | 0.2 | 0.2 | 0.5 | 5.0 | 1.0 | >1 | 0.1 | 0.03 | 2 | 0.1 | 0.02 |
| Et 729 | <1 | 0.2 | 0.2 | 0.5 | 5.0 | 2.5 | >1 | 0.2 | 0.02 | 1.5 | 0.05 | 0.08 |
| Et 815 | 2.5 | 2.5 | 5.0 | 5.0 | nt | 5.0 | — | >1 | 0.1 | — | 5 | 0.75 |
| Et 759B | nt^{e} | 5.0 | 5.0 | 5.0 | 10 | 2.5 | >1 | 0.7 | 0.5 | — | >1 | 3.90 |
| Et 745B | 2.5 | 5.0 | 10 | 10 | nt | 2.5 | — | >1 | 0.5 | — | 3 | nt |
| Et 759C | | 1.0 | 2.5 | 2.5 | nt | 2.5 | 2.5 | — | >1 | 0.5 | >5 | 0.1 |
| Et 745 | | 10 | 20 | 2.5 | 50 | 50 | — | >1 | 0.3 | — | 5 | 6.50 |
| Et 731 | nt | 100 | 100 | 100 | 200 | 200 | >1 | — | — | — | — | 6.20 |
| Et 736 | | 0.5 | 1.0 | 2.5 | 2.5 | 2.5 | 0.5 | 0.4 | 0.1 | — | 0.5 | 0.38 |
| Et 722 | | 1.0 | 1.0 | 2.0 | 2.0 | 5.0 | 0.9 | 0.4 | 0.1 | >1 | 0.5 | 0.70 |
| Et 808 | nt | nt | nt | nt | nt | nt | nt | nt | nt | nt | nt | nt |
| Et 597 | nt | 2.0 | 2.0 | 2.0 | 2.0 | 2.5 | 0.7 | 0.08 | 0.01 | — | 0.25 | 0.14 |
| Et 583 | nt | 10 | 10 | 10 | 5.0 | 25 | 1.0 | 1.0 | 0.4 | — | 0.5 | 0.74 |
| Et 594 | nt | 10 | 20 | 25 | 25 | 25 | 0.8 | 0.5 | 0.5 | — | 1.0 | 0.37 |
| Et 743 deriv. | | | | | | | | | | | | |
| 6′-Ac, 15-Br | | 1.0 | 2.4 | 2.5 | nt | 2.5 | — | 0.5 | — | 5 | 0.42 | nt |
| 5-deAc, 21-CN | nt | 0.25 | 1.0 | 1.0 | nt | 2.5 | >1 | 0.2 | 0.09 | >5 | 1.0 | 0.32 |
| Et 729 deriv. | | | | | | | | | | | | |
| N-CHO | nt | | | | | | — | — | — | — | 4 | 6.60 |
| N-CHO, 15-Br (18) | nt | 50 | 200 | 200 | nt | 250 | — | — | — | — | — | nt |

^{a}Cell lines: L1210 = murine lymphoma cells; P388 = murine lymphoma cells; A549 = human lung carcinoma; HT29 = human colon carcinoma; MEL28 = human carcinoma; CV-1 = monkey kidney cells.
^{b}Prot. = protein synthesis inhibition; DNA = DNA synthesis inhibition; RNA = RNA synthesis inhibition.
^{c}DNAp = DNA polymerase inhibition; RNAp = RNA polymerase inhibition.
^{d}Bacillus subtilis.
^{e}nt = not tested.

Crude Et 596 (as a single major peak by FABMS in the m/z 500-800 region, see Figure A) exhibited antimicrobial activity against B. subtilis at 0.3 μg/disc (MIC).

The present invention will be further illustrated with reference to the following examples which aid in the understanding of the present invention, but which are not to be construed as limitations thereof. All percentages reported herein, unless otherwise specified, are percent by weight. All temperatures are expressed in degrees Celsius.

A. General Extraction Procedure: Preparation of Fraction A.

This procedure is a typical example for the extraction of a frozen specimen of E. turbinata.

### EXAMPLE A-I

A total of 102 kg of the tunicate was extracted separately in three batches. Frozen tunicate (30 kg) was soaked with 2-propanol (16 L) for 12 h, keeping the temperature below 4° C. The extract was agitated and the alcoholic extract was filtered through a large mesh cooking sieve. The extract was stored in a freezer (−20° C.) pending concentration. The residual tissue was extracted three or four times with 4 L of solvent, then squeezed to give a cake (10% of original weight of the tunicate). The extract stored in the freezer was concentrated to an aqueous emulsion by rotary evaporator, using a dry-ice trap and high vacuum pump. This emulsion was extracted by EtOAc until the green color disappeared from the aqueous layer. The organic extract Was concentrated to give an oil (25 g, combined with the other batches, 41 g) which was partitioned between the lower and the upper layers of MagicSolvent (7:4:4:3, EtOAc-heptane-MeOH—H₂O). The lower layer was concentrated to afford an active solid (4.4 g, 14-mm inhibition zone at 10 μg against B. subtilis), which was separated on a C-18 flash column (Fuji-Davison gel, 60 g) into four fractions. The first (bright orange color) and the second (pale yellow to yellow-green color) fractions were eluted with MeOH-aq-NaCl (0.2M), 9:2, the third fraction (dark green) was eluted with MeOH and finally the column was washed with MeOH—CHCl₃ (elution volumes may vary but the color of the fraction is indicative). FABMS and TLC (9:1 CHCl₃—MeOH, silica) of the above fractions were monitored to evaluate the quality of the samples. TLC and FABMS of the first fraction (Fraction A) showed the presence of mainly Et743-type compounds while those of the second fraction showed the presence of Et 736-type compounds.

### EXAMPLE A-II

This example was the extraction procedure employed for tunicate samples shipped from Puerto Rico in September, 1992, labeled “fresh” and “stored”. These samples were separately processed for comparison. A sample (fresh, 2.8 Kg) was extracted with 2-propanol (4 L, less than 5° C.) for 10 h. The alcoholic extract was decanted and residual solid was extracted twice (2-propanol, 1 L each). Alcoholic extracts were combined and concentrated to give an aqueous emulsion (2.5 L). This emulsion was extracted with EtOAc (1 L×1, 0.5 L×1). The organic layer was concentrated and then partitioned between the lower and upper layers of MagicSolvent (200 mL). The upper layer was separated by C18 (25 g) flash chromatography. The first eluent (MeOH-aq-NaCl, 0.4 M, 9:2, 50 mL from the solvent front) afforded active Fraction A 1 (89.3 mg), and the second fraction (wash with MeOH—CHCl₃) gave mostly lipids (116.5 mg). Fraction A1 was flash-chromatographed over silica gel (pre-treated with NH₃, 0.5% w/w). The first (9:1 MeOH—CHCl₃ eluate) and the second (4:1 MeOH—CHCl₃ eluate) fractions exhibited activity against B. subtilis (12 mm zone at 0.3 μg/disc).

B. Separation of Fraction A

Several different approaches have been employed for the separation of Fraction A.

### EXAMPLE B-I

Fraction A (890 mg) was separated by HSCCC using the solvent system (CH₂Cl₂-toluene-MeOH—H₂O, 15:15:23:7). The upper phase was used as stationary phase (2400 mL of the solvent prepared gave 1000 mL of lower layer).

The following operating conditions were used: flow rate 1.9 mL/min; counter balance-brass×3+aluminum×3; rotation speed 600 rpm; 15 mL/fraction. Each fraction was monitored by TLC and FABMS. The results are shown in Table B-1 below.

TABLE B-I
HSCCC of Fraction A-Example B-I

| Tube # | Fraction # | weight, mg. | Components (Et's FABMS) |
|---|---|---|---|
| 1-2 | RS9-34-1 | 5.8 | NR^{a} |
| 3-4 | RS9-34-2 | 69.2 | 736 |
| 5-6 | RS9-34-3 | 19.8 | 736, 722, 640, 626 |
| 7-8 | RS9-34-4 | 29.3 | 770, 626, 722, 744 |
| 9-12 | RS9-34-5 | 45.2 | 759, 626, 722 |
| 13-14 | RS9-34-6 | 12.8 | 722, 745, 752, 759, 768 |
| 15-18 | RS9-34-7 | 27.4 | 745 |
| 19-23 | RS9-34-8 | 51.1 | 745, 743 |
| 24-29 | RS9-34-9 | 62.6 | 745, 743 |
| 30-34 | RS9-34-10 | 82.1 | 743, 759, 775 |
| 35-40 | RS9-34-11 | 109.0 | 743, 759, 775, 792 |
| stationary | RS9-23-12 | 353.7 | 729, 743, 761, 775 |
| phase | | | |

^{a}NR = not recorded

### EXAMPLE B-II

Fraction A (1.08 g) was separated by a flash silica gel column (treated with NH₃ before use, 0.5% w/w). The first fraction eluted with CCHCl₃:MeOH (6:1) contained Et's (669 mg) which were separated by HSCCC using the same conditions as above except the lower layer was used as stationary phase and each 22 mL/tube was collected (Table B-II).

This process was repeated to separate the rest of Fraction A (1.03 g).

TABLE B-II
HSCCC of Fraction A-Example B-II

| Tube # | Fraction # | weight, mg. | Components (FABMS) |
|---|---|---|---|
| 1-7 | RS9-36-1 | 51.8 | NR^{a} |
| 8-11 | RS9-36-2 | 11.3 | NR |
| 12-13 | RS9-36-3 | 28.2 | NR |
| 14-18 | RS9-36-4 | 14.7 | NR |
| 19-20 | RS9-36-5 | 76.3 | NR |
| 21-25 | RS9-36-6 | 19.7 | NR |
| 26 | RS9-36-7 | 69.5 | 729, 745 |
| 27 | RS9-36-8 | 5.1 | 743, 745 |
| 28-35 | RS9-36-9 | 123.9 | 745, 743 |
| 36-40 | RS9-36-10 | 24.3 | 743 |
| 41-48 | RS9-36-11 | 99.0 | contains Et |
| | | | 736 & 722 |
| 49-54 | RS9-36-12 | 32.9 | same as above |
| | | | 722 |
| stationary | RS9-36-13 | 129.0 | same as above |
| phase | | | |

^{a}NR = not recorded

After the above HSCCC separation, the known ecte inascidins in each fraction could easily be monitored by TLC and FABMS. Each selected fraction was ready to be separated to give individual Et's.

### EXAMPLE B-III

Fraction A prepared by Dr. Ignacio Manzanares at PharmaMar S.A. (“IMCL-2”, 80 mg) was separated by HSCCC (conditions: solvent toluene:Et₂O:MeOH:H₂O, 6:6:6:3; lower layer mobile; flow rate 1.8 mL/min).

TABLE B-III
HSCCC of IMCL2

| Fraction # | weight, mg. | Components (FABMS) |
|---|---|---|
| Et-12-1 | 9.9 | Et 597, 583, 628 |
| Et-12-2 | 7.2 | Et 597, 628, 583, 570 |
| Et-12-3 | 8.0 | Et 597, 628, 580 |
| Et-12-4 | 8.5 | Et 597, 580, 745 |
| Et-12-5 | 14.5 | Et 597, 628, 730, 745 |
| Et-12-6 | 9.9 | Et 628 |
| Et-12-7 | 4.0 | Et 743, 745 |
| Et-12-8 | 5.4 | Et 627, 584, 771 |
| Et-12-9 | 1.7 | non-Et |

Fraction RS 2-12-6. (Example B-III) was separated by HPLC (MeOH-0.04 M NeCl, 3:1) to afforded a fraction (0.5 mg) containing mainly Et 596.

C. Separation of Ecteinascidins. Example C-L.

Isolation of Et 808

Fractions containing mainly Et's 736 and 722 (by FABMS)—RS 9-36-12-14, 9-38-10-11, 9-40-7 (757 mg)—were combined, then separated by HSCCC (CCl₄:CHCl₃:MeOH:EtOAc:CH₃CN:H₂O, (2:3:5:5:-2.5:3; lower layer mobile phase) as follows:

TABLE C-L

| Tube # | Fraction # | weight, mg. | Components (FABMS) |
|---|---|---|---|
| 1-3 | RS9-44-1 | 150.2 | amino alcohols? |
| 4 | RS9-44-2 | 114.5 | Et 736, 625, 753 |
| 5 | RS9-44-3 | 74.2 | Et 722 |
| 6 | RS9-44-4 | 44.4 | Et 722 |
| 7 | RS9-44-5 | 34.6 | Et 722, 808 |
| 8-42 | RS9-44-6-12 | — | — |

Fraction RS 9-44-5 was combined with RS 9-34-4. (above) and separated by a silica gel column (15:1, CHCl₃:MeOH) then HPLC (C18, MeOH:CH₃CN:aq-NaCl, 0.4 mL, 3:4:1) to give pure Et 808 (0.81 mg, tr=10.2 min.)

### EXAMPLE C-II

Isolation of Et 745B and 731

Fractions containing mainly Et 729 (by FABMS)-ORS 9-36-7, 9-38-6-7, 9-40-7 (182 mg—were combined then separated by HSCCC (toluene:Et₂O:MeOH:H₂O: 10:10:10:5, lower layer mobile phase) as follows:

TABLE C-II

| Tube # | Fraction # | weight, mg. | Components (FABMS) |
|---|---|---|---|
| 1-2 | RS9-47-1 | 30.2 | Et 729, 731 |
| 3 | RS9-47-2 | 7.4 | Et 729, 731 |
| 4 | RS9-47-3 | 11.3 | Et 729, 731 |
| 5-10 | RS9-47-4 | 44.4 | Et 729, 745B |
| 11-14 | RS9-47-5 | 51.7 | Et 729, 731 |

Fraction RS 9-47-4 was separated by a flash silica gel column (CHCl₃-MeOH: 12:1) to give a mixture of Et 729 and 745 (29 mg) and semipure Et 745B (12.4 mg). Et 745B was separated-by HPLC (C18, MeOH:ammonium formate, 0.02 M, 4:1). The fraction containing Et 745 (single peak) was concentrated to dryness and the residue was triturated by CH₂Cl₂ to give pure Et 745B (6 mg).

RS 9-47-5 was separated on a flash silica gel column (CHCl₃:MeOH, 12:1) to give semipure. Et 729 (38 mg) and Et 731, which was purified by RPHPLC (3:1, MeOH:NaCl, 0.02 M) to give pure Et 731 (2.8 mg).

### EXAMPLE C-III

### Separation of Et 815

Fractions containing Et 743, RS 9-34-11, 9-36-11 and 9-38-9 (292 mg)—were combined then separated by silica gel flash column chromatography (CHCl₃:MeOH, 12:1). Fractions were combined by TLC as follows:

TABLE C-III

| Fraction # | weight, mg. | Components (FABMS) |
|---|---|---|
| RS9-48-1 | 30.5 | Et 743 |
| RS9-48-2 | 88.1 | Et 743 |
| RS9-48-3 | 39.5 | Et 729, 743, 745, 815 |
| RS9-48-4 | 31.3 | Et Yellow |
| RS9-48-5 | 14.1 | Et Yellow |
| RS9-48-6 | 38.0 | fats |

Fractions RS 9-48-3 was separated on a flash silica gel column (CHCl₃:MeOH, 18:1) then by RPHPLC (MeOH:NaCl, 0.02 M: 3:1) to give mainly four fractions. The first and second fractions (Et 1-13-1 and −2, 1.9 and 3.2 mg, respectively) were combined then separated on a silica gel column (1.5×25 cm column, CHCl₃:MeOH, 6:1) to give pure Et 597 (Et 2-14-1, 1.45 mg) and Et 583 (Et 2-14-2, 1.43 mg).

Purification of Et 594

Et 2-12-8 was purified by RPHPLC (same conditions as in preceding paragraph). A broad peak (tR=33-42 min) gave Et-594 (1.2 mg).

Physical Data of the New Et's

Ecteinascidin 731: a light brown solid; [α]_{D}²⁵ −100° (c 0.49, MeOH); ¹H NMR (500 MHz, CD₃OD) δ 6.54 (1H, s), 6.42 (1H, s), 6.37 (1H, s), (1H, d, J=1.0 Hz), 5.92 (1H, d, J=1.0 Hz), 5.05 (1H, d, J=11.0 Hz), 4.45 (1H, br), 4.43 (1H, d, J=4.5 Hz), 3.69 (3H, s), 3.56 (3H, s), 3.26 (1H, dd, J=10.5, 2.0 Hz), 2.58 (1H, dd, J=2.5, 10.5 Hz), 2.23 (3H, s), 2.11 (3H, s), 1.98 (3H, s);

¹³C NMR (CDCl₃—CD₃OD, 2:1) δ 172.80, 169.45, 147.15, 145,73, 145.59, 143.44, 141.56, 140.49, 131.67, 130.43, 128.38, 125.58, 123.65, 121.84, 120.95, 115.37, 115.17, 113.40, 110.84, 102.22, 64.57, 64.34, 61.47, 60.18, 59.10, 48.05, 46.17, 42.78, 41.69, 39.55, 29.66, 28.19, 20.48, 15.89, 9.77; negative ion FABMS m/z 730 (M−H)⁻.

Anal. Calcd for C₃₈H₄₂N₃O₁₀S (M+H)⁺; Mr 732.2591. Found Mr 732.2606 (HRFABMS).

Ecteinascidin 745B: a light brown solid; [α]_{D}²⁵ −196° (c 0.60, MeOH); ¹H NMR (300 MHz, CD₃OD—CDCl₃, 2:1) δ 6.61 (1H, s), 6.42 (1H, s), 6.20 (1H, brs), 6.06 (1H, d, J=1.0 Hz), 6.00 (1H, d, J=1.0 Hz), 4.74 (2H, m, H,22a, 11), 4.68 (1H, s, H-1), 4.22 (1H, dd, J=11.4, 1.5 Hz, H-22b), 3.97 (1H, d, J=2.4 Hz, H-3); 3.77 (1H, brd, J=4.8 Hz, H-13), 3.72 (3H, s), 3.57 (3H,s), 3.11-2.88 (2H, m), 2.85-2.70 (2H, m), 2.65-2.55 (1H, m), 2.48-21.38 (1H, m), 2.25 (3H, s), 2.23 (3H, s), (3H, s), 2.15 (1H, brd J=13.5 Hz, H-12′), 2.01 (3H, s); ¹³C NMR (125 MHz, CD₃OD-CDCl₃, 1:1) δ 172.57 s, 170.26 s, 147.19 s, 146.86 s, 146.37 s, 146.24 s, 145.79s, 142.69 s, 141.66 s, 131.36 s, 131.29 s, 129.29 s, 124.42 s, 123.63 s, 122.45 d, 120.91 s, 115.69 d, 113.83 s, 110.64 d, 103.01 t, 90.51 d, 71.25 d, 68.55 t, 62.32 s, 61.98, b 60.37 b, 58.23 d, 56.61 d, 55.45 d, 47.66 d, 46.20 d, 40.37 t, 29,05 t, 28.04 t, 20.82 q, 16.09 q, 10.48 q; negative ion FABMS m/z 776 (M+MeOH−H)⁻.

Anal. Calcd for C₃₈H₄₀N₃O₁₁S (M+H−H₂O): Mr 746.2384. Found: Mr 746.2398 (HRFABMS),

Ecteinascidin 815: a light yellow solid; [α]_{D}² −131° (c 0.358, MeOH); ¹H NMR (500 MHz, CDCl₃); δ 9.24 (1H, s), 8.07 (1H, s), 6.70 (1H, s), 6.47 (1H, s), 6.44 (1H, s), 5.97 (1H, s), 5.93 (1H, s), 5.37 (1H, d, J=11.5 Hz, H-22a), 3.60 (3H, s), 3.48 (3H, s), 2.35 (6H, s), 2.25 (3H, s), 2.00 (3H, s); ¹³C NMR (125 MHz, CD₃OD) δ 193.38 d (CHO), 188.56 d (CHO), 149.95 s (C-18), 146.25 s (C-7), 146.21 s (C-6′), 146.10 s (C-7′), 144.89 s (C-17) 141.64 s (C-5), 140.97 s (C-8), 133.32 s (C-20), 129.94 s (C-16), 128.26 (C-10′), 124.68 (C-9′), 120.62 (C-10), 120.43 d (C-15), 115.90 s (C-19), 115.68 (C-9),.115.29 d (C-5′), 114.54 (C-6), 110.95 d (C-8′), 102.64 t (O—CH₂—O), 65.09 s (C-1′), 60.25 q (OCH₃), 59.40 d (C-3), 58.79 d (C-1), 58.32 d (C-21′), 56.67 d (C-11), 55.53 q (OCH₃), 55.42 d, (C-13), 42.93 d (C-4), 42.28 t (c-3′), 42.21 t (C-12′), 39.12 q (NCH₃), 28 t (C-4′), 27.79 t (C-14), 20.39 q (5Ac), 16.12 q (CH₃-16), 9.81 q (CH₃-6); negative ion FABMS m/z 814 (M−H)⁻.

Anal. Calcd for C₄₂H₄₆N₃O₁₂S (M+H): Mr 816.28.02. Found: Mr 816.2788 (HRFABMS).

Ecteinascidin 808: a light brown solid; [α]_{D}²⁵ −110° (c 0.081, MeOH); ¹H NMR (500 MHz, CD₃OD—CDCl₃-10:1); δ 9.02 (1H, s), 8.36 (1H, s), 7.32 (1H, d, J=8.0 Hz), 7.22 (1H, d, J=8.5 Hz), 7.00 (1H, ddd, J=8.0, 7.0, 1.5), 6.91 (1H, ddd, J=7.5, 7.0, 0.5), 6.70 (1H, s), 6.21 (1H, d, J=1.0), 6.03 (1H, d, J=1.0), 5.38 (1H, d, J=11.5 Hz), 4.95 (1H, d, J=3.5 Hz), 4.67 (1H, brs), 4.58 (1H, brs), 4.06 (1H, brs), 4.03 (1 H, dd, J=11.50, 2.0), 3.77 (3H,s), 3.72 (1H, brs), 3.23 (1H, m), 2.90 (1H, m), 2.75 (1H, d, J=15.0 Hz), 2.63 (2H, m), 2.53 (3H, s), 2.39 (3H, s), 2.28 (3H, s), 2.00 (3H, s).

Anal. Calcd for C₄₃H₄₅N₄O₁₆S (M+H): Mr 809.2856. Found: Mr 809.2851 (HRFABMS).

Ecteinascidin 596: (insufficient sample); m/z 629 as a methanol adduct; HRFABMS m/z 629.2171.

Ecteinascidin 597: a light brown solid, decomposed slowly in solution giving reddish color; [α]_{D}²⁵ −49° (c 0.17, MeOH); UV (λₘₐₓ) 207 (ε 46000), 230 (sh, 15000), 278 (3800); ¹H NMR (500 MHz, CD₃OD), see Table I.

Anal. Calcd for C₃₀H₃₆N₃O₈S (M+H−H₂O): Mr 598.2223. Found: Mr 598.2219 (HRFABMS).

Ecteinascidin 583: a light yellow solid; [α]_{D}²² −470 (c 0.1 4, CHCl₃-MeOH, 6:1); UV (λₘₐₓ) 207 (ε 48000), 230 (sh, 9200), 280 (2100), 290 (2300); ¹H NMR (500 MHz, CD₃Cl—CD₃OD, 3:1), see Table I.

Anal. Calcd for C₂₉H₃₄N₃O₈S (M+H−H₂O): Mr 584.2066. Found: Mr 584.2054 (HRFABMS).

Ecteinascidin 594: a light yellow solid; [α]_{D}²² −58° (c 1.1, MeOH); (λₘₐₓ) 207 (ε 60500), 230 (sh, 11000), 287 (2900); ¹H NMR (500 MHz, CD₃OD), see Table I; FABMS (glycerol matrix in the presence of oxalic acid and water) m/z 627 (M+MeOH, magic bullet matrix),. 595 (M+H), 613 (M+H₂O), 687 (M+glycerol).

Anal. Calcd for C₃₀H₃₁N₂O₉S (M+H); Mr 595.1750. Found: Mr 595.1716 (HRFABMS).

Preparation of N-Acetyl Ecteinascidin 597:

Et 597 (1 mg. Et 1-33-1) was treated with Ac₂O (50 mL) and Et₃N (5 μL) at room temperature for 30 min. The product was passed through a Sep-pak silica gel column with CHCl₃-MeOH (9:1) then purified by RPHPLC (9:2: MeOH:NaCl, 0.04 M) to give a monoacetyl derivative (0.5 mg): ¹H NMR (CDCl₃) δ 6.70 (1H, s), 5.48 (1H, brm), 5.12 (1H, d, J=12.0 Hz), 5.10 (1H, brs), 4.87 (1H, brs), 4.53 (1H, m), 4.32 (1H, dd, J=11.5, 2 Hz), 4.22 (1H, brd, J=2.5 Hz), 4.00 (1H, brd, J=8.5 Hz), 3.82,(3H, s), 3.80 (3H, s), 3.47 (1H, d, J=18.5 Hz), 3.10 (1H, dd, J=18.5 Hz), 2.58 (3H, s), 2.36 (3H, s), 2.27 (3H, s), 2.08 (3H, s), 1.87 (3H, s); FABMS m/z 641 (M+H−H₂O).

Anal. Calcd for C₃₂H₃₉N₃O₉S (M+H−H₂O): Mr 641.2407. Found: Mr 641.2398 (HRFABMS).

A small amount of diacetyl derivative (only enough to take FABMS data) was also isolated.

Anal. Calcd for C₃₄H₄₁N₃O₁₀S (M+H−H₂O): Mr 683.2513; Found: Mr 683.2492 (HRFABMS).

The following literature references have been cited herein, and each is hereby incorporated herein by reference:
1. (a) Rinehart, K. L. et al., J. Nat. Prod., 53: 771-791 (1990); (b) Wright, A. E. et al., J. Org. Chem., 55: 4508-4512 (1990); and (c) Sakai, R., Ph.D., Dissertation, University of Illinois, Urbana, 1991.
2. Sakai et al., Proc. Nat. Acad. Sci. U. S.A., 89: 11456-11460 (1992).
3. Dr. D. Lednicer, National Cancer Institute, Division of Cancer Treatment, Bethesda, Md., and Dr. G. Faircloth, PharmaMar, Inc., Cambridge, Mass., personal communications.
4. Rinehart et al., J. Org. Chem., 55: 4512-4515.(1990).

The present invention has been described in detail, including the preferred embodiments thereof. However, it will be appreciated that those skilled in the art, upon consideration of the present disclosure, may make modifications and/or improvements on this invention and still be within the scope and spirit of this invention.

1. Substantially pure Ecteinascidin 731, free of cellular debris of Ecteinascidia turbinata and having the following physical characteristics: light brown solid; [α]_{D}²⁵ −100° (c 0.49, MeOH); ¹H NMR (500 MHz, CD₃OD) δ 6.54 (1H, s), 6.42 (1H, s), 6.37 (1H, s), (1H, d, J=1.0 Hz), 5.92 (1H, d, J=1.0 Hz), 5.05 (1H, d, J=11.0 Hz), 4.45 (1H, br), 4.43 (1H, d, J=4.5 Hz), 3.69 (3H, s), 3.56 (3H, s), 3.26 (1H, dd, J=10.5, 2.0 Hz), 2.58 (1H, dd, J=2.5, 10.5 Hz), 2.23 (3H, s), 2.11 (3H, s), 1.98 (3H, s); ¹³C NMR (CDCl₃—CD₃OD, 2:1) δ 172.80, 169.45, 147.15, 145.73, 145.59, 143.44, 141.56, 140.49, 131.67, 130.43, 128.38, 125.58, 123.65, 121.84, 120.95, 115.37, 115.17, 113.40, 110.84, 102.22, 64.57, 64.34, 61.47, 60.18, 59.10, 48.05, 46.17, 42.78, 41.69, 39.55, 29.66, 28.19, 20.48, 15.89, 9.77; negative ion FABMS m/z 730 (M−H)⁻; Anal. Found Mr 732.2606 (HRFABMS).

2-24. (canceled)

25. Substantially pure Ecteinascidin 815, free of cellular debris of Ecteinascidia turbinata and having the following physical characteristics: light yellow solid; [.alpha.].sub.D.sup.25−131.degree. (c 0.358, MeOH); .sup.1H NMR (500 MHz, CDCl.sub.3); .delta. 9.24 (1H, S), 8.07 (1H, s), 6.70 (1H, s), 6.47 (1H, s), 6.44 (1H, s), 5.97 (1H, s), 5.93 (1H, s), 5.37 (1H, d, J=11.5 Hz, H-22a), 3.60 (3H, s), 3.48 (3H, s), 2.35 (6H, s), 2.25 (3H, s), 2.00 (3H, s); .sup.13C NMR (125 MHz, CD.sub.3OD) .delta. 193.38 d (CHO), 188.56 d (CHO), 149.95 s (C-18), 146.25 s (C-7), 146.21 s (C-6′), 146.10 s (C-7′), 144.89 s (C-17) 141.64 s (C-5), 140.97 s (C-8), 133.32 s (C-20), 129.94 s (C-16), 128.26 (C-10′), 124.68 (C-9′), 120.62 (C-10), 120.43 d (C-15), 115.90 s (C-19), 115.68 (C-9), 115.29 d (C-5′), 114.54 (C-6), 110.95 d (C-8′), 102.64 t (O—CH.sub.2-O), 65.09 s (C-1′), 60.25 q (OCH.sub.3), 59.40 d (C-3), 58.79 d (C-1), 58.32 d (C-21′), 56.67 d (C-11), 55.53 q (OCH.sub.3), 55.42 d, (C-13), 42.93 d (C-4), 42.28 t (c-3′), 42.21 t (C-12′), 39.12 q (NCH.sub.3), 28 t (C-4′), 27.79 t (C-14), 20.39 q (5 Ac), 16.12 q (CH3-16), 9.81 q (CH3-6); negative ion FABMS m/z 814 (M−H).sup.-; Anal. Calcd for C.sub.42H.sub.46N.sub.3O.sub.12S (M+H): Mr 816.28.02; Found: Mr 816.2788 (HRFABMS).

26. Substantially pure Ecteinascidin 808, free of cellular debris of Ecteinascidia turbinata and having the following physical characteristics: light brown solid; [.alpha.].sub.D.sup.25−110.degree. (c 0.081, MeOH); .sup.1H NMR (500 MHz, CD.sub.30D-CDCl.sub.3-10:1); delta. 9.02 (1H, s), 8.36 (1H, s), 7.32 (1H, d, J=8.0 Hz), 7.22 (1H, d, J=8.5 Hz), 7.00 (1H, ddd, J=8.0, 7.0, 1.5), 6.91 (1H, ddd, J=7.5, 7.0, 0.5), 6.70 (1H, s), 6.21 (1H, d, J=1.0), 6.03 (1H, d, J=1.0), 5.38 (1H, d, J=11.5 Hz), 4.95 (1 Hz d, J=3.5 Hz), 4.67 (1H, brs), 4.58 (1H, brs), 4.06 (1H, brs), 4.03 (1H, dd, J-11.50, 2.0), 3.77 (3H, s), 3.72 (1H, brs), 3.23 (1H, m), 2.90 (1H, m), 2.75 (1H, d, J=15.0 Hz), 2.63 (2H, m), 2.53 (3H, s), 2.39 (3H, s), 2.28 (3H, s), 2.00 (3H, s); Anal. Calcd for C.sub.43H.sub.45N.sub.40.sub.10S (M+H): Mr 809.2856. Found: Mr 809.2851 (HRFABMS).

27. Substantially pure Ecteinascidin 594, free of cellular debris of Ecteinascidia turbinata and having the following physical characteristics: light yellow solid; [.alpha.].sub.D.sup.22−58.degree. (c 1.1, MeOH); (.lambda..sub.max) 207 (.epsilon. 60500), 230 (sh, 11000), 287 (2900); sup.1H NMR (500 MHz, CD.sub.30D), see Table I; FABMS (glycerol matrix in the presence of oxalic acid and water) m/z 627 (M+MeOH, magic bullet matrix), 595 (M+H), 613 (M+H.sub.2O), 687 (M+glycerol); Anal. Calcd for C.sub.30H.sub.31N.sub.2O.sub.9S (M+H); Mr 595.1750; Found: Mr 595.1716 (HRFABMS).

28. A pharmaceutical or veterinary composition comprising an effective antitumor amount of the compound according to claim 1 and a pharmaceutically acceptable carrier, diluent or excipient, wherein the tumor is selected from the group consisting of mammalian leukemia, mammalian melanoma and mammalian lung carcinoma.

29. A pharmaceutical or veterinary composition comprising an effective antitumor amount of the compound according to claim 25 and a pharmaceutically acceptable carrier, diluent or excipient, wherein the tumor is selected from the group consisting of mammalian leukemia, mammalian melanoma and mammalian lung carcinoma.

30. A pharmaceutical or veterinary composition comprising an effective antitumor amount of the compound according to claim 26 and a pharmaceutically acceptable carrier, diluent or excipient, wherein the tumor is selected from the group consisting of mammalian leukemia, mammalian melanoma and mammalian lung carcinoma.

31. A pharmaceutical or veterinary composition comprising an effective antitumor amount of the compound according to claim 27 and a pharmaceutically acceptable carrier, diluent or excipient, wherein the tumor is selected from the group consisting of mammalian leukemia, mammalian melanoma and mammalian lung carcinoma.

32. A method of treating a patient suffering from a mammalian tumor selected from the group consisting of mammalian leukemia, mammalian melanoma and mammalian lung carcinoma. comprising administering to said patient, an effective antitumor amount of the compound according to claim 1 and a pharmaceutically acceptable carrier, diluent or excipient.

33. A method of treating a patient suffering from a mammalian tumor selected from the group consisting of mammalian leukemia, mammalian melanoma and mammalian lung carcinoma, comprising administering to said patient, an effective antitumor amount of the compound according to claim 25 and a pharmaceutically acceptable carrier, diluent or excipient.

34. A method of treating a patient suffering from a mammalian tumor selected from the group consisting of mammalian leukemia, mammalian melanoma and mammalian lung carcinoma, comprising administering to said patient, an effective antitumor amount of the compound according to claim 26 and a pharmaceutically acceptable carrier, diluent or excipient.

35. A method of treating a patient suffering from a mammalian tumor selected from the group consisting of mammalian leukemia, mammalian melanoma and mammalian lung carcinoma, comprising administering to said patient, an effective antitumor amount of the compound according to claim 27 and a pharmaceutically acceptable carrier, diluent or excipient.
